SEQUENCE LISTING (34 sequences)

SEQ ID NO: 1 - protein, 120 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSS

SEQ ID NO: 2 - protein, 111 aa, synthetic construct
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELK

SEQ ID NO: 3 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 4 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 5 - protein, 14 aa, synthetic construct
GGGGSGGGGSGGGG

SEQ ID NO: 6 - protein, 15 aa, synthetic construct
GGGGSGGGGSGGGGS

SEQ ID NO: 7 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 8 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 9 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 10 - protein, 107 aa, synthetic construct [sequence omitted]

SEQ ID NO: 11 - protein, 105 aa, synthetic construct
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 13 - protein, 150 aa, Homo sapiens [sequence omitted]

SEQ ID NO: 14 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 15 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 16 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 17 - protein, 218 aa, synthetic construct
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 20 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 450 aa, synthetic construct; identical to SEQ ID NO: 20

SEQ ID NO: 22 - protein, 615 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 23 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 24 - protein, 615 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 25 - protein, 615 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 26 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 27 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 28 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 29 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 30 - protein, 614 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGDCDIEGKDGKQYESVLMVSIDQLLDSMKEIGSNCLNNEFNFFKRHICDANKEGMFLFRAARKLRQFLKMNSTGNFDLHLLKVSEGTTILLNCTGQVKGRKPAALGEAQPTKSLEENKSLKEQKKLNDLCFLKRLLQEIKTCWNKILMGTKEH

SEQ ID NO: 31 - protein, 614 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGDCDIEGKDGKQYESVLMVSIDQLLDSMKEIGSNCLNNEFNFFKRHICDANKEGMFLFRAARKLRQFLKMNSTGNFDLHLLEVSEGTTILLNCTGQVKGRKPAALGEAQPTKSLEENKSLKEQKKLNDLCFLKRLLQEIKTCWNKILMGTKEH

SEQ ID NO: 32 - protein, 218 aa, synthetic construct; identical to SEQ ID NO: 18

SEQ ID NO: 33 - protein, 449 aa, synthetic construct; identical to SEQ ID NO: 19

SEQ ID NO: 34 - protein, 615 aa, synthetic construct; identical to SEQ ID NO: 22

(19) United States
(12) Patent Application Publication
(10) Pub. No.: US 2024/0269277 A1
(43) Pub. Date: Aug. 15, 2024

(54) ALTERNATIVE PD1-IL7V IMMUNOCONJUGATES FOR THE TREATMENT OF CANCER

(71) Applicant: Hoffmann-La Roche Inc., Little Falls, NJ (US)

(72) Inventors: Laura CODARRI DEAK, Au (CH); Greta DURINI, Zürich (CH); Anne FREIMOSER-GRUNDSCHOBER, Zürich (CH); Christian KLEIN, Bonstetten (CH); Laura LAUENER, Baden (CH); Ekkehard MOESSNER, Kreuzlingen (CH); Valeria NICOLINI, Erlenbach (CH); Cindy SCHULENBURG, Zürich (CH); Pablo UMAÑA, Lachen (CH)

(73) Assignee: Hoffmann-La Roche Inc., Little Falls, NJ (US)

(21) Appl. No.: 18/634,666

(22) Filed: Apr. 12, 2024

Related U.S. Application Data
(63) Continuation of application No. PCT/EP2022/078327, filed on Oct. 12, 2022.

(30) Foreign Application Priority Data
Oct. 14, 2021 (EP) 21202552.2

(51) Int. Cl. A61K 39/395; A61K 38/20; C07K 14/54; C07K 16/28

(52) U.S. Cl. CPC A61K 39/39558; A61K 38/2046; C07K 14/5418; C07K 16/2818; C07K 2317/76; C07K 2317/92; C07K 2319/33

(57) ABSTRACT

Provided are immunoconjugates for use in the treatment of cancer, particularly wherein the immunoconjugates comprise a mutant interleukin 7 polypeptide and an antibody that binds to PD-1. In addition, provided are the use of such immunoconjugates and methods of treating cancer comprising administering said immunoconjugates.

### CROSS REFERENCE TO RELATED APPLICATIONS

This application is a continuation of International Application No. PCT/EP2022/078327, filed Oct. 12, 2022, which claims priority to European Patent Application No. 21202552.2 filed Oct. 14, 2021 both of which are incorporated herein by reference in its entirety.

### SEQUENCE LISTING

This application contains a Sequence Listing which has been submitted electronically in XML format and is hereby incorporated by reference in its entirety. Said XML copy, created on Apr. 11, 2024, is named P37163-US.xml and is 40.6 KB bytes in size.

### FIELD OF THE INVENTION

The present invention generally relates to immunoconjugates for use in the treatment of cancer, particularly immunoconjugates comprising a mutant interleukin-7 polypeptide and an antibody that binds to PD-1. In addition, the invention relates to the use of such immunoconjugates and methods of treating cancer comprising administering said immunoconjugates.

### BACKGROUND

Interleukin-7 (IL-7) is a cytokine mainly secreted by stromal cells in lymphoid tissues. It is involved in the maturation of lymphocytes, e.g. by stimulating the differentiation of multipotent hematopoetic stem cells to lymphoblasts. IL-7 is essential for T-cell development and survival, as well as for mature T-cell homeostasis. A lack of IL-7 causes immature immune cell arrest (Lin J. et al. (2017), Anticancer Res. 37(3):963-967).

IL-7 binds to the IL-7 receptor, which is composed of the IL-7R alpha chain (IL-7Rα, CD127) as well as the common gamma chain (γc, CD132, IL-2Rγ), that is mutual to the interleukines IL-2, IL-4, IL-7, IL-9, IL-15 and IL-21 (Rochman Y. et al., (2009) Nat Rev Immunol. 9:480-490). Whereas γc is expressed by most haematopoietic cells, IL-7Rα is almost exclusively expressed by cells of the lymphoid lineage (Mazzucchelli R. and Durum S. K. (2007) Nat Rev Immunol. 7(2): 144-54). IL-7Rα is found on the surface of T cells across their differentiation from naïve to effector while its expression is reduced on terminally differentiated T cells and is virtually absent from the surface of regulatory T cells. IL-7Rα mRNA and protein expression levels are negatively regulated by IL-2, therefore IL-7Rα is downregulated in recently activated T cells expressing the IL-2Rα (CD25) (Xue H. H, et al. 2002, PNAS. 99(21): 13759-64), this mechanism ensures the IL-2 mediated rapid clonal expansion of recently primed T cells while IL-7 role is to equally maintain all T cell clones. IL-7Rα has also been recently described on a newly characterized precursor population of CD8 T cells, TCF-1+PD-1+stem-like CD8 T cells, which is found in the tumor of cancer patients responding to PD-1 blockade (Hudson et al., 2019, Immunity 51, 1043-1058; Im et al., PNAS, vol. 117, no. 8, 4292-4299; Siddiqui et al., 2019, Immunity 50, 195-211: Held et al., Sci. , Transl. Med. 11: eaay6863 (2019): Vodnala and Restifo, Nature, Vol 576, 19/26 December 2019). Although, until today, there are no scientific descriptions of the effect of IL-7 on the stem like CD8 T cells, IL-7 could be used to expand this population of tumor reactive T cells in order to increase the number of patients responding to check point inhibitors.

IL-7, IL-7Rα and γc form a ternary complex, which signals over the JAK/STAT (Janus kinase (JAK)-signal transducer and activator of transcription (STAT)) pathway as well as the PI3K/Akt (Phosphatidylinositol 3-kinase (PI3K), serine/threonine protein kinase, protein kinase B (AKT)) signaling cascade, leading to the development and homeostasis of B- and T-cells (Niu N. and Qin X. (2013) Cell Mol Immunol. 10(3): 187-189, Jacobs et al., (2010), J Immunol. 184(7): 3461-3469).

IL-7 is a 25 kDa 4-helix bundle, monomeric protein. The helix length varies from 13 to 22 amino acids, which is similar to the helix length of other common gamma chain (γc, CD132, IL-2Rγ) binding interleukines. However, IL-7 shows a unique turn motif in the A helix, which was shown to stabilize the IL-7/IL-7Rα interaction (McElroy, C. A. et al., (2009) Structure 17: 54-65). Whereas the A helix interacts with both receptor chains IL-7Rα and γc, the C helix interacts predominantly with IL-7Rα and the D helix with the γc chain (sequence and structural alignments based on PDB:3DI2 and PDB:2ERJ). Variant IL-7s with modifications to reduce heterogeneity and/or reduced affinity/potency have been described in WO 2020/127377 A1 and WO 2020/236655 A1.

Programmed cell death protein 1 (PD-1 or CD279) is an inhibitory member of the CD28 family of receptors, that also includes CD28, CTLA-4, ICOS and BTLA. PD-1 is a cell surface receptor and is expressed on activated B cells, T cells, and myeloid cells (Okazaki et al (2002) Curr. Opin. Immunol. 14: 391779-82: Bennett et al. (2003) J Immunol 170:711-8). The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Two ligands for PD-1 have been identified, PD-L1 and PD-L2, that have been shown to downregulate T cell activation upon binding to PD-1 (Freeman et al (2000) J Exp Med 192: 1027-34; Latchman et al (2001) Nat Immunol 2:261-8: Carter etal (2002) Eur J Immunol 32:634-43). Both PD-L1 and PD-L2 are B7 homologs that bind to PD-1, but do not bind to other CD28 family members. One ligand for PD-1, PD-L1 is abundant in a variety of human cancers (Dong et al (2002) Nat. Med 8:787-9). The interaction between PD-1 and PD-L1 results in a decrease in tumor infiltrating lymphocytes, a decrease in T-cell receptor mediated proliferation allowing immune evasion by the cancerous cells (Dong et al. (2003) J. Mol. Med. 81:281-7: Blank et al. (2005) Cancer Immunol. Immunother. 54:307-314: Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat 7. Acad. ScL USA 99: 12293-7: Brown et al. (2003) J. Immunol. 170:1257-66).

Antibodies that bind to PD-1 are described e.g. in WO 2017/055443 A1. Approved therapeutic PD-1 antibodies include pembrolizumab and nivolumab.

### SUMMARY OF THE INVENTION

The present invention provides a novel approach of targeting a mutant form of IL-7 with advantageous properties for immunotherapy directly to immune effector cells, such as cytotoxic T lymphocytes, rather than tumor cells, through conjugation of the mutant IL-7 polypeptide to an antibody that binds to PD-1. This results in cis-delivery of the IL-7 mutant to PD-1 expressing immune subsets, especially tumor reactive T cells e.g. CD8+ PD1+ TCF+ T cell subsets and their progeny.

The IL-7 mutants used in the present invention have been designed to overcome the problems associated with cytokine immunotherapy, in particular toxicity caused by the induction of VLS, tumor tolerance caused by the induction of AICD, and immunosuppression caused by activation of T_{reg} cells. In addition to circumventing escape of tumors from tumor-targeting as mentioned above, targeting of the IL-7 mutant to immune effector cells may further increase the preferential activation of tumor specific CTLs over immunosuppressive T_{reg} cells due to lower PD-1 and IL-7Rα expressing levels on Tregs than CTLs. By using an antibody that binds to PD-1, the suppression of T-cell activity induced by the interaction of PD-1 with its ligand PD-L1 may additionally be reversed, thus further enhancing the immune response.

The invention provides an immunoconjugate for use in the treatment of cancer, wherein the immunoconjugate comprises (i) a mutant IL-7 polypeptide and (ii) and antibody, wherein the antibody binds to PD1 and comprises the VH domain according to SEQ ID NO: 1 and the VL domain according to SEQ ID NO: 2: and wherein the mutant IL-7 polypeptide comprises an amino acid substitution at the position of G85 of human IL-7 according to SEQ ID NO: 14, wherein the amino acid substitution reduces the binding affinity of the mutant interleukin-7 polypeptide to IL-7Rα compared to an interleukin-7 polypeptide comprising SEQ ID NO: 14.

Further the invention provides the use of an immunoconjugate in the manufacture of a medicament for the treatment of cancer, wherein the immunoconjugate comprises (i) a mutant IL-7 polypeptide and (ii) and antibody, wherein the antibody binds to PD1 and comprises the VH domain according to SEQ ID NO: 1 and the VL domain according to SEQ ID NO: 2: and wherein the mutant IL-7 polypeptide comprises an amino acid substitution at the position of G85 of human IL-7 according to SEQ ID NO: 14, wherein the amino acid substitution reduces the binding affinity of the mutant interleukin-7 polypeptide to IL-7Rα compared to an interleukin-7 polypeptide comprising SEQ ID NO: 14.

Further the invention provides a method of treating cancer in an individual comprising administering to said individual a therapeutically effective amount of an immunoconjugate, wherein the immunoconjugate comprises (i) a mutant IL-7 polypeptide and (ii) and antibody, wherein the antibody binds to PD1 and comprises the VH domain according to SEQ ID NO: 1 and the VL domain according to SEQ ID NO: 2: and wherein the mutant IL-7 polypeptide comprises an amino acid substitution at the position of G85 of human IL-7 according to SEQ ID NO: 14, wherein the amino acid substitution reduces the binding affinity of the mutant interleukin-7 polypeptide to IL-7Rα compared to an interleukin-7 polypeptide comprising SEQ ID NO: 14.

In one aspect, the amino acid substitution at the position of G85 of human IL-7 according to SEQ ID NO: 14 is G85E.

In a further aspect, mutant interleukin-7 polypeptide further comprises an amino acid substitution at position K81. In one aspect, the mutant interleukin-7 polypeptide comprises the amino acid substitution K81E.

In one aspect, immunoconjugate comprises not more than one mutant IL-7 polypeptide.

In another aspect, the antibody comprises an Fc domain composed of a first and a second subunit. In one aspect, the Fc domain is an IgG class, particularly an IgG1 subclass, Fc domain. In a further aspect, the Fc domain is a human Fc domain. In one aspect, the antibody is an IgG class, particularly an IgG1 subclass immunoglobulin.

In another aspect, the Fc domain comprises a modification promoting the association of the first and the second subunit of the Fc domain. 13. In one aspect, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. In another aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In yet a further aspect, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

In one aspect, the mutant IL-7 polypeptide is fused at its amino-terminal amino acid to the carboxy-terminal amino acid of one of the subunits of the Fc domain, particularly the first subunit of the Fc domain, optionally through a linker peptide. In one aspect, the linker peptide has the amino acid sequence of SEQ ID NO: 5.

In another aspect, the Fc domain comprises one or more amino acid substitution that reduces binding to an Fc receptor, particularly an Fcγ receptor, and/or effector function, particularly antibody-dependent cell-mediated cytotoxicity (ADCC). In another aspect, said one or more amino acid substitution is at one or more position selected from the group consisting of L234, L235, and P329 (Kabat EU index numbering). In one aspect, each subunit of the Fc domain comprises the amino acid substitutions L234A, L235A and P329G (Kabat EU index numbering). In one aspect, the immunoconjugate for use, the use or the method according to the invention, comprises a polypeptide comprising an amino acid sequence that is at least about 80%, 85%, 90%, 95%, 96%, 97%, 98%, 99% or 100% identical to the sequence of SEQ ID NO: 17, a polypeptide comprising an amino acid sequence that is at least about 80%, 85%, 90%, 95%, 96%, 97%, 98%, 99% or 100% identical to the sequence of SEQ ID NO: 19, and a polypeptide comprising an amino acid sequence that is at least about 80%, 85%, 90%, 95%, 96%, 97%, 98%, 99% or 100% identical to a sequence selected from the group consisting of SEQ ID NO: 24 and SEQ ID NO: 25.

### BRIEF DESCRIPTION OF THE DRAWINGS

FIG. 1: Schematic representation of an IgG-IL-7 immunoconjugate format, comprising two Fab domains (variable domain, constant domain), a heterodimeric Fc domain and a mutant IL-7 polypeptide fused to a C-terminus of the Fc domain.

FIG. 2: IL-7R signaling (STAT5-P) in co-cultured PD1 pre-blocked and PD1⁺ CD4 T cells upon treatment with PD1(alt)-IL7wt in comparison to PD1(alt)-IL7 VAR21 and PD1(alt)-IL7 VAR18/VAR21. IL-7R signaling (STAT5-P) depicted as frequency of STAT5-P in co-cultured PD1⁺ (solid line) and PD-1 pre-blocked (dotted line) CD4 T cells after 12 min upon exposure. Mean±SEM of 3 donors.

FIG. 3: IL-7R signaling (STAT5-P) in co-cultured PD1 pre-blocked and PD1⁺ CD4 T cells upon treatment with Reference molecules 5-8 in comparison to PD1(alt)-IL7 VAR21 and PD1(alt)-IL7 VAR18/VAR21. IL-7R signaling (STAT5-P) depicted as frequency of STAT5-P in co-cultured PD1⁺ (solid line) and PD-1 pre-blocked (dotted line) CD4 T cells after 12 min upon exposure. Mean±SEM of 3 donors.

FIG. 4: IL-7R signaling (STAT5-P) in co-cultured PD1 pre-blocked and PD1⁺ CD4 T cells upon treatment with Reference molecules 9 and 10 in comparison to PD1(alt)-IL7 VAR21 and PD1(alt)-IL7 VAR18/VAR21. IL-7R signaling (STAT5-P) depicted as frequency of STAT5-P in co-cultured PD1⁺ (solid line) and PD-1 pre-blocked (dotted line) CD4 T cells after 12 min upon exposure. Mean±SEM of 3 donors.

### DETAILED DESCRIPTION OF THE INVENTION

### Definitions

The immunoconjugates disclosed herein can be particularly useful in the uses and methods described herein.

Terms are used herein as generally used in the art, unless otherwise defined in the following.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g. reduced binding to IL-7Rα and/or IL-2Rγ. Amino acid sequence deletions and insertions include amino- and/or carboxy-terminal deletions and insertions of amino acids. An example of a terminal deletion is the deletion of the residue in position 1 of full-length human IL-7. Preferred amino acid mutations are amino acid substitutions. For the purpose of altering e.g. the binding characteristics of an IL-7 polypeptide, non-conservative amino acid substitutions, i.e. replacing one amino acid with another amino acid having different structural and/or chemical properties, are particularly preferred. Preferred amino acid substitions include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1: 1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

IL-7 binds to the IL-7 receptor, which is composed of the IL-7R alpha chain (also refered to as IL-7Ralpha, IL-7Rα, IL7Rα, IL-7a, IL7Rα or CD127 herein) as well as the common gamma chain (also refered to as γc, CD132, IL-2Rgamma, IL-2Rg. IL2Rg. IL-2Rγ or IL2Rγ herein), that is mutual to the interleukines IL-2, IL-4, IL-7, IL-9, IL-15 and IL-21 (Rochman Y. et al., (2009) Nat Rev Immunol. 9:480-490).

The affinity of the mutant or wild-type IL-7 polypeptide for the IL-7 receptor can be determined in accordance with the method set forth in the WO 2012/107417 by surface plasmon resonance (SPR), using standard instrumentation such as a BIAcore instrument (GE Healthcare) and receptor subunits such as may be obtained by recombinant expression (see e.g. Shanafelt et al., Nature Biotechnol 18, 1197-1202 (2000)). Alternatively, binding affinity of IL-7 mutants for the IL-7 receptor may be evaluated using cell lines known to express one or the other such form of the receptor. Specific illustrative and exemplary embodiments for measuring binding affinity are described hereinafter.

The term “interleukin-7” or “IL-7” as used herein, refers to any native IL7 from any vertebrate source, including mammals such as primates (e.g. humans) and rodents (e.g., mice and rats), unless otherwise indicated. The term encompasses unprocessed IL-7 as well as any form of IL-7 that results from processing in the cell. The term also encompasses naturally occurring variants of IL-7, e.g. splice variants or allelic variants. The amino acid sequence of an exemplary human IL-7 is shown in SEQ ID NO: 14.

The term “IL-7 mutant” or “mutant IL-7 polypeptide” as used herein is intended to encompass any mutant forms of various forms of the IL-7 molecule including full-length IL-7, truncated forms of IL-7 and forms where IL-7 is linked to another molecule such as by fusion or chemical conjugation. “Full-length” when used in reference to IL-7 is intended to mean the mature, natural length IL-7 molecule. For example, full-length human IL-7 refers to a molecule that has a polpypetide sequence according to SEQ ID NO: 14. The various forms of IL-7 mutants are characterized in having a at least one amino acid mutation affecting the interaction of IL-7 with IL7Ralpha and/or IL2Rgamma. This mutation may involve substitution, deletion, truncation or modification of the wild-type amino acid residue normally located at that position. Mutants obtained by amino acid substitution are preferred. Unless otherwise indicated, an IL-7 mutant may be referred to herein as a mutant IL-7 peptide sequence, a mutant IL-7 polypeptide, a mutant IL-7 protein, a mutant IL-7 analog or a IL-7 variant.

Designation of various forms of IL-7 is herein made with respect to the sequence shown in SEQ ID NO: 14. Various designations may be used herein to indicate the same mutation. For example a mutation from Valine at position 15 to Alanine can be indicated as 15A, A15, A₁₅, V15A, or Val15Ala.

By a “human IL-7 molecule” as used herein is meant an IL-7 molecule comprising an amino acid sequence that is at least about 90%, at least about 91%, at least about 92%, at least about 93%, at least about 94%, at least about 95% or at least about 96% identical to the human IL-7 sequence of SEQ ID NO:14. Particularly, the sequence identity is at least about 95%, more particularly at least about 96%. In particular embodiments, the human IL-7 molecule is a full-length IL-7 molecule.

As used herein, a “wild-type” form of IL-7 is a form of IL-7 that is otherwise the same as the mutant IL-7 polypeptide except that the wild-type form has a wild-type amino acid at each amino acid position of the mutant IL-7 polypeptide. For example, if the IL-7 mutant is the full-length IL-7 (i.e. IL-7 not fused or conjugated to any other molecule), the wild-type form of this mutant is full-length native IL-7. If the IL-7 mutant is a fusion between IL-7 and another polypeptide encoded downstream of IL-7 (e.g. an antibody chain) the wild-type form of this IL-7 mutant is IL-7 with a wild-type amino acid sequence, fused to the same downstream polypeptide. Furthermore, if the IL-7 mutant is a truncated form of IL-7 (the mutated or modified sequence within the non-truncated portion of IL-7) then the wild-type form of this IL-7 mutant is a similarly truncated IL-7 that has a wild-type sequence. For the purpose of comparing IL-7 receptor binding affinity, IL-7 receptor binding or biological activity of various forms of IL-7 mutants to the corresponding wild-type form of IL-7, the term wild-type encompasses forms of IL-7 comprising one or more amino acid mutation that does not affect IL-7 receptor binding compared to the naturally occurring, native IL-7. In certain embodiments according to the invention the wild-type IL-7 polypeptide to which the mutant IL-7 polypeptide is compared comprises the amino acid sequence of SEQ ID NO: 14.

By “regulatory T cell” or “T_{reg} cell” is meant a specialized type of CD4⁺ T cell that can suppress the responses of other T cells, called peripheral tolerance. T_{reg} cells are characterized by elevated expression of the α-subunit of the IL-2 receptor (CD25), low or absent IL-7Rα (CD127) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. As used herein, the term “effector cells” refers to a population of lymphocytes which survival and/or homeostasis are affected by IL-7. Effector cells include memory CD4+ and CD8+ cells and recently primed T cells including tumor reactive stem-like T cells.

As used herein, the term “PD1”, “human PD1”, “PD-1” or “human PD-1” (also known as Programmed cell death protein 1, or Programmed Death 1) refers to the human protein PD1 (SEQ ID NO: 7, protein without signal sequence)/(SEQ ID NO: 8, protein with signal sequence). See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a KD value of the binding affinity for binding to human PD-1 of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). The KD value of the binding affinity may be determined in a Surface Plasmon Resonance assay using the Extracellular domain (ECD) of human PD-1 (PD-1-ECD, see SEQ ID NO: 13) as antigen.

By “specific binding” is meant that the binding is selective for the antigen and can be discriminated from unwanted or non-specific interactions. The ability of an antibody to bind to a specific antigen (e.g. PD-1) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody comprised in an immunoconjugate for use 30) according to the invention to an unrelated protein is less than about 10% of the binding of the antibody to the antigen as measured, e.g., by SPR. The antibody comprised in the immunoconjugate for use as described herein specifically binds to PD-1.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W. Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448: W. R. Pearson (1996) “Effective protein sequence comparison” Meth. Enzymol. 266:227- 258: and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10: ext: −2: Ktup=2) to ensure a global, rather than local, alignment is performed. Percent amino acid identity is given in the output alignment header.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the polypeptides for use according to present invention, as well as positive and negative strand forms, and double-stranded forms. Isolated polynucleotides or nucleic acids further include such molecules produced synthetically. In addition, a polynucleotide or a nucleic acid may be or may include a regulatory element such as a promoter, ribosome binding site, or a transcription terminator.

“Isolated polynucleotide (or nucleic acid) encoding [e.g. an immunoconjugate of the invention]” refers to one or more polynucleotide molecules encoding antibody heavy and light chains and/or IL-7 polypeptides (or fragments thereof), including such polynucleotide molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. The expression cassette may comprise polynucleotide sequences that encode immunoconjugates for use according to the invention or fragments thereof.

The term “vector” or “expression vector” refers to a DNA molecule that is used to introduce and direct the expression of a specific gene to which it is operably associated in a cell. The term includes the vector as a self-replicating nucleic acid structure as well as the vector incorporated into the genome of a host cell into which it has been introduced. The expression vector may comprise an expression cassette. Expression vectors allow transcription of large amounts of stable mRNA. Once the expression vector is inside the cell, the ribonucleic acid molecule or protein that is encoded by the gene is produced by the cellular transcription and/or translation machinery. The expression vector may comprise an expression cassette that comprises polynucleotide sequences that encode immunoconjugates for use according to the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the immunoconjugates of the present invention. Host cells include cultured cells, e.g. mammalian cultured cells, such as HEK cells, CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen binding activity.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment, i.e. that is not in its natural milieu. No particular level of purification is required. For example, an isolated antibody can be removed from its native or natural environment. Recombinantly produced antibodies expressed in host cells are considered isolated for the purpose of the invention, as are native or recombinant antibodies which have been separated, fractionated, or partially or substantially purified by any suitable technique. As such, the immunoconjugates for use according to the present invention are isolated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Holliger and Hudson, Nature Biotechnology 23:1126-1136 (2005). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097: WO 1993/01161; Hudson et al., Nat Med 9, 129-134 (2003): and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA: see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (lgG₁), γ₂ (lgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W. H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)):
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)):
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)): and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. Such variable domains are referred to herein as “humanized variable region”. A humanized antibody optionally may comprise at least a portion of an antibody constant region derived from a human antibody. In some embodiments, some FR residues in a humanized antibody are substituted with corresponding residues from a non-human antibody (e.g., the antibody from which the HVR residues are derived), e.g., to restore or improve antibody specificity or affinity. A “humanized form” of an antibody, e.g. of a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain embodiments, a human antibody is derived from a non-human transgenic mammal, for example a mouse, a rat, or a rabbit. In certain embodiments, a human antibody is derived from a hybridoma cell line. Antibodies or antibody fragments isolated from human antibody libraries are also considered human antibodies or human antibody fragments herein.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447. numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprised in an immunoconjugate for use according to the invention, comprises an additional C-terminal glycine-lysine dipeptide (G446 and K447, numbering according to EU index of Kabat). In one embodiment of the invention, a heavy chain including a subunit of an Fc domain as specified herein, comprised in an immunoconjuate for use according to the invention, comprises an additional C-terminal glycine residue (G446, numbering according to EU index of Kabat). Compositions for use according to the invention, such as the pharmaceutical compositions described herein, comprise a population of immunoconjugates for use according to the invention. The population of immunoconjugates may comprise molecules having a full-length heavy chain and molecules having a cleaved variant heavy chain. The population of immunoconjugates may consist of a mixture of molecules having a full-length heavy chain and molecules having a cleaved variant heavy chain, wherein at least 50%, at least 60%, at least 70%, at least 80% or at least 90% of the immunoconjugates have a cleaved variant heavy chain. In one embodiment of the invention, a composition comprising a population of immunoconjugates for use according to the invention comprises an immunoconjugate comprising a heavy chain including a subunit of an Fc domain as specified herein with an additional C-terminal glycine-lysine dipeptide (G446 and K447. numbering according to EU index of Kabat). In one embodiment of the invention, a composition comprising a population of immunoconjugates for use according to the invention comprises an immunoconjugate comprising a heavy chain including a subunit of an Fc domain as specified herein with an additional C-terminal glycine residue (G446. numbering according to EU index of Kabat). In one embodiment of the invention, such a composition comprises a population of immunoconjugates for use according to the invention comprised of molecules comprising a heavy chain including a subunit of an Fc domain as specified herein: molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” when used in reference to antibodies refers to those biological activities attributable to the Fc region of an antibody, which vary with the antibody isotype. Examples of antibody effector functions include: Clq binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

“Reduced binding”, for example reduced binding to an Fc receptor or CD25, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

As used herein, the term “immunoconjugate” refers to a polypeptide molecule that includes at least one IL-7 molecule and at least one antibody. The IL-7 molecule can be joined to the antibody by a variety of interactions and in a variety of configurations as described herein. In particular embodiments, the IL-7 molecule is fused to the antibody via a peptide linker. Particular immunoconjugates for use according to the invention essentially consist of one IL-7 molecule and an antibody joined by one or more linker sequences.

By “fused” is meant that the components (e.g. an antibody and an IL-7 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, immunoconjugates of the invention are used to delay development of a disease or to slow the progression of a disease.

### DETAILED DESCRIPTION OF THE EMBODIMENTS

### Mutant IL-7 Polypeptide

The IL-7 variants of the immunoconjugates for use according to the present invention have advantageous properties for immunotherapy.

The mutant interleukin-7 (IL-7) polypeptide of the immunoconjugates for use according to the invention comprises at least one amino acid mutation that reduces affinity of the mutant IL-7 polypeptide to the α-subunit of the IL-7 receptor and/or the IL-2Rγ subunit.

Mutants of human IL-7 (hIL-7) with decreased affinity to IL-7Rα and/or IL-2Rγ may for example be generated by amino acid substitution at amino acid position 81 or 85 or combinations thereof (numbering relative to the human IL-7 sequence SEQ ID NO: 14). Exemplary amino acid substitutions include K81E and G85E. In one embodiment the mutant interleukin-7 (IL-7) polypeptide of a immunoconjugate for use according to the invention comprises an amino acid substituion at position G85 of human IL-7 according to SEQ ID NO: 14. In one embodiment said mutant interleukin-7 (IL-7) polypeptide comprises the amino acid substituion G85E according to SEQ ID NO: 14. In a further embodiment said mutant interleukin-7 (IL-7) polypeptide of the immunoconjugates for use comprises amino acid substituions at positions K81 and G85 of human IL-7 according to SEQ ID NO: 14. In one embodiment said mutant interleukin-7 (IL-7) polypeptide comprises the amino acid substituions K81E and G85E according to SEQ ID NO: 14.

The mutant interleukin-7 (IL-7) polypeptide may comprise at least one amino acid mutation that improves the homonogeneity of the polypeptide, preferably in one of the amino acid positions 93 and 118 or combinations thereof. Exemplary amino acid substitutions include T93A and S118A.

In some embodimenets of the invention the mutant interleukin-7 polypeptide comprises an amino acid sequence of SEQ ID NO: 15. In some embodimenets of the invention the mutant interleukin-7 polypeptide comprises an amino acid sequence of SEQ ID NO:16. These mutants exhibit substantially reduced affinity to the interleukin 7 receptor compared to a wild-type form of the IL-7 mutant.

Other characteristics of IL-7 mutants as disclosed herein include reduced affinity to IL-7Rα to allow PD-1 mediated delivery of IL-7 in cis (on the same cell) on PD-1 expressing CD4 T cells, compared to wild-type IL-7 which is mainly delivered in trans (on cell in close proximity) when in a PD1-IL-7 immunoconjugate.

In certain embodiments said amino acid mutation reduces the affinity of the mutant IL-7 polypeptide to the IL-Rα and/or the IL-2Rγ by at least 5-fold, specifically at least 10-fold, more specifically at least 25-fold.

Reduction of the affinity of IL-7 for the IL-7Rα and/or the IL-2Rγ in combination with elimination of the N-glycosylation of IL-7 results in an IL-7 protein with improved properties. For example, elimination of the N-glycosylation site results in a more homogenous product when the mutant IL-7 polypeptide is expressed in mammalian cells such as CHO or HEK cells. Elimination of N-glycosylation sites of IL-7 can be achieved by amino acid mutations at a position corresponding to residue 72, 93 or 118 of human IL-7.

In a specific embodiment, the mutant IL-7 polypeptide of the immunoconjugate for use according to the invention can still elicit one or more of the cellular responses selected from the group consisting of: proliferation in T lymphocyte cells, effector functions in an primed T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment, the mutant IL-7 polypeptide of the immunoconjugate for use according to the invention comprises no more than 12, no more than 11, no more than 10, no more than 9, no more than 8, no more than 7, no more than 6, or no more than 5 amino acid mutations as compared to the corresponding wild-type IL-2 sequence, e.g. the human IL-7 sequence of SEQ ID NO: 14. In a particular embodiment, the mutant IL-7 polypeptide of the immunoconjugate for use according to the invention comprises no more than 5 amino acid mutations as compared to the corresponding wild-type IL-7 sequence, e.g. the human IL-7 sequence of SEQ ID NO: 14.

### Immunoconjugates

Immunoconjugates as described herein comprise an IL-molecule and an antibody. Such immunoconjugates significantly increase the efficacy of IL-7 therapy by directly targeting IL-7 e.g. into a tumor microenvironment. According to the invention, an antibody comprised in the immunoconjugate for use can be a whole antibody or immunoglobulin, or a portion or variant thereof that has a biological function such as antigen specific binding affinity.

The general benefits of immunoconjugate therapy are readily apparent. For example, an antibody comprised in an immunoconjugate recognizes a tumor-specific epitope and results in targeting of the immunoconjugate molecule to the tumor site. Therefore, high concentrations of IL-7 can be delivered into the tumor microenvironment, thereby resulting in activation and proliferation of a variety of immune effector cells mentioned herein using a much lower dose of the immunoconjugate than would be required for unconjugated IL-7. However, this characteristic of IL-7 immunoconjugates may again aggravate potential side effects of the IL-7 molecule: Because of the significantly longer circulating half-life of IL-7 immunoconjugate in the bloodstream relative to unconjugated IL-7, the probability for IL-7 or other portions of the fusion protein molecule to activate components generally present in the vasculature is increased. The same concern applies to other fusion proteins that contain IL-7 fused to another moiety such as Fc or albumin, resulting in an extended half-life of IL-7 in the circulation. Therefore immunoconjugates comprising a mutant IL-7 polypeptide as described herein with reduced toxicity compared to wild-type forms of IL-7, is particularly advantageous.

As described hereinabove, targeting IL-7 directly to immune effector cells rather than tumor cells may be advantageous for IL-7 immunotherapy.

Accordingly, the invention provides a mutant IL-7 polypeptide as described hereinbefore, and an antibody that binds to PD-1 for use in the treatment of cancer. In one embodiment said mutant IL-7 polypeptide and antibody form a fusion protein, i.e. the mutant IL-7 polypeptide shares a peptide bond with the antibody. In some embodiments, the antibody comprises an Fc domain composed of a first and a second subunit. In a specific embodiment the mutant IL-7 polypeptide is fused at its amino-terminal amino acid to the carboxy-terminal amino acid of one of the subunits of the Fc domain, optionally through a linker peptide. In some embodiments, the antibody is a full-length antibody. In some embodiments, the antibody is an immunoglobulin molecule, particularly an IgG class immunoglobulin molecule, more particularly an IgG₁ subclass immunoglobulin molecule. In one such embodiment, the mutant IL-7 polypeptide shares an amino-terminal peptide bond with one of the immunoglobulin heavy chains. In certain embodiments the antibody is an antibody fragment. In some embodiments the antibody is a Fab molecule or a scFv molecule. In one embodiment the antibody is a Fab molecule. In another embodiment the antibody is a scFv molecule. The immunoconjugate may also comprise more than one antibody. Where more than one antibody is comprised in the immunoconjugate, e.g. a first and a second antibody, each antibody can be independently selected from various forms of antibodies and antibody fragments. For example, the first antibody can be a Fab molecule and the second antibody can be a scFv molecule. In a specific embodiment each of said first and said second antibodies is a scFv molecule or each of said first and said second antibodies is a Fab 20) molecule. In a particular embodiment each of said first and said second antibodies is a Fab molecule. In one embodiment each of said first and said second antibodies binds to PD-1.

### Immunoconjugate Formats

Exemplary immunoconjugate formats are described in PCT publication no. WO 2011/020783, which is incorporated herein by reference in its entirety. These immunoconjugates comprise at 5 least two antibodies. Thus, in one embodiment, the immunoconjugate for use according to the present invention comprises a mutant IL-7 polypeptide as described herein, and at least a first and a second antibody. In a particular embodiment, said first and second antibody are independently selected from the group consisting of an Fv molecule, particularly a scFv molecule, and a Fab molecule. In a specific embodiment, said mutant IL-7 polypeptide shares an amino- or carboxy-terminal peptide bond with said first antibody and said second antibody 30 shares an amino- or carboxy-terminal peptide bond with either i) the mutant IL-7 polypeptide or ii) the first antibody. In a particular embodiment, the immunoconjugate for use according to the invention consists essentially of a mutant IL-7 polypeptide and first and second antibodies. particularly Fab molecules. joined by one or more linker sequences. Such formats have the advantage that they bind with high affinity to the target antigen (PD-1), but provide only monomeric binding to the IL-7 receptor, thus avoiding targeting the immunoconjugate to IL-7 receptor bearing immune cells at other locations than the target site. In a particular embodiment. a mutant IL-7 polypeptide shares a carboxy-terminal peptide bond with a first antibody. particularly a first Fab molecule, and further shares an amino-terminal peptide bond with a second antibody, particularly a second Fab molecule. In another embodiment, first antibody, particularly a first Fab molecule, shares a carboxy-terminal peptide bond with a mutant IL-7 polypeptide, and further shares an amino-terminal peptide bond with a second antibody, particularly a second Fab molecule. In another embodiment, a first antibody, particularly a first Fab molecule, shares an amino-terminal peptide bond with a first mutant IL-7 polypeptide, and further shares a carboxy-terminal peptide with a second antibody, particularly a second Fab molecule. In a particular embodiment, a mutant IL-7 polypeptide shares a carboxy-terminal peptide bond with a first heavy chain variable region and further shares an amino-terminal peptide bond with a second heavy chain variable region. In another embodiment a mutant IL-7 polypeptide shares a carboxy-terminal peptide bond with a first light chain variable region and further shares an amino-terminal peptide bond with a second light chain variable region. In another embodiment, a first heavy or light chain variable region is joined by a carboxy-terminal peptide bond to a mutant IL-7 polypeptide and is further joined by an amino-terminal peptide bond to a second heavy or light chain variable region. In another embodiment, a first heavy or light chain variable region is joined by an amino-terminal peptide bond to a mutant IL-7 polypeptide and is further joined by a carboxy-terminal peptide bond to a second heavy or light chain variable region. In one embodiment, a mutant IL-7 polypeptide shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a mutant IL-7 polypeptide and further shares an amino-terminal peptide bond with a second Fab heavy or light chain. In other embodiments, a first Fab heavy or light chain shares an amino-terminal peptide bond with a mutant IL-7 polypeptide and further shares a carboxy-terminal peptide bond with a second Fab heavy or light chain. In one embodiment, the immunoconjugate for use according to the invention comprises a mutant IL-7 polypeptide sharing an amino-terminal peptide bond with one or more scFv molecules and further sharing a carboxy-terminal peptide bond with one or more scFv molecules.

Particularly suitable formats for the immunoconjugates for use according to the present invention, however comprise an immunoglobulin molecule as antibody. Such immunoconjugate formats are described in WO 2012/146628, which is incorporated herein by reference in its entirety.

Accordingly, in particular embodiments, the immunoconjugate for use according to the invention comprises a mutant IL-7 polypeptide as described herein and an immunoglobulin molecule that binds to PD-1, particularly an IgG molecule, more particularly an IgGi molecule. In one embodiment the immunoconjugate comprises not more than one mutant IL-7 polypeptide. In one embodiment the immunoglobulin molecule is human. In one embodiment, the immunoglobulin molecule comprises a human constant region, e.g. a human CH1, CH2, CH3 and/or CL domain. In one embodiment, the immunoglobulin comprises a human Fc domain, particularly a human IgG₁ Fc domain. In one embodiment the mutant IL-7 polypeptide shares an amino- or carboxy-terminal peptide bond with the immunoglobulin molecule. In one embodiment, the immunoconjugate essentially consists of a mutant IL-7 polypeptide and an immunoglobulin molecule, particularly an IgG molecule, more particularly an IgG1 molecule, joined by one or more linker sequences. In a specific embodiment the mutant IL-7 polypeptide is fused at its amino-terminal amino acid to the carboxy-terminal amino acid of one of the immunoglobulin heavy chains, optionally through a linker peptide.

The mutant IL-7 polypeptide may be fused to the antibody directly or through a linker peptide, comprising one or more amino acids, typically about 2-20 amino acids. Linker peptides are known in the art and are described herein. Suitable, non-immunogenic linker peptides include, for example, (G₄S)ₙ, (SG₄)ₙ, (G₄S)ₙ or G₄(SG₄)ₙ linker peptides. “n” is generally an integer from 1 to 10, typically from 2 to 4. In one embodiment the linker peptide has a length of at least 5 amino acids, in one embodiment a length of 5 to 100, in a further embodiment of 10 to 50 amino acids. In a particular embodiment, the linker peptide has a length of 15 amino acids. In one embodiment the linker peptide is (GxS)ₙ or (GxS)ₙGₘ with G=glycine, S=serine, and (x=3, n=3, 4, 5 or 6, and m=0), 1, 2 or 3) or (x=4, n=2, 3, 4 or 5 and m=0, 1, 2 or 3), in one embodiment x=4 and n=2 or 3, in a further embodiment x=4 and n=3, in a further embodiment x=4, n=2 and m=4. In a particular embodiment the linker peptide is (G₄S)₂G₄ (SEQ ID NO: 5). In one embodiment, the linker peptide has (or consists of) the amino acid sequence of SEQ ID NO: 5. An alternative linker peptide comprising the amino acid sequence of SEQ ID NO: 6 may be used.

In a particular embodiment, the immunoconjugate for use according to the invention comprises a mutant IL-7 molecule and an immunoglobulin molecule, particularly an IgG₁ subclass immunoglobulin molecule, that binds to PD-1, wherein the mutant IL-7 molecule is fused at its amino-terminal amino acid to the carboxy-terminal amino acid of one of the immunoglobulin heavy chains through the linker peptide of SEQ ID NO: 5.

In a particular embodiment, the immunoconjugate for use according to the invention comprises a mutant IL-7 molecule and an antibody that binds to PD-1, wherein the antibody comprises an Fc domain, particularly a human IgG1 Fc domain, composed of a first and a second subunit, and the mutant IL-7 molecule is fused at its amino-terminal amino acid to the carboxy-terminal amino acid of one of the subunits of the Fc domain through the linker peptide of SEQ ID NO: 5.

### PD-1 Antibodies

The antibody comprised in the immunoconjugate of the invention binds to PD-1, particularly human PD-1, and is able to direct the mutant IL-7 polypeptide to a target site where PD-1 is expressed, particularly to a T cell that expresses PD-1, for example associated with a tumor.

Suitable PD-1 antibodies that may be used in the immunoconjugate for the treatment of cancer, the use or the methof of treatment according to the invention comprise the VH domain and the VL domain of therapeutic PD-1 antibodies, e.g. of pembrolizumab or nivolumab.

The immunoconjugate for use according to the invention may comprise two or more antibodies, which may bind to the same or to different antigens. In particular embodiments, however, each of these antibodies binds to PD-1. In one embodiment, the antibody comprised in the immunoconjugate for use according to the invention is monospecific. In a particular embodiment, said immunoconjugate comprises a single, monospecific antibody, particularly a monospecific immunoglobulin molecule.

The antibody can be any type of antibody or fragment thereof that retains specific binding to PD-1, particularly human PD-1. Antibody fragments include, but are not limited to, Fv molecules, scFv molecule, Fab molecule, and F(ab′)₂ molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG₁ subclass immunoglobulin.

In some embodiments, the antibody is a monoclonal antibody.

In some embodiments, the antibody comprises a heavy chain variable region (VH) comprising an amino acid sequence that is at least about 95%, 96%, 97%, 98%, 99% or 100% identical to the amino acid sequence of SEQ ID NO:1. In some embodiments, the antibody comprises a light chain variable region (VL) comprising an amino acid sequence that is at least about 95%, 96%, 97%, 98%, 99% or 100% identical to the amino acid sequence of SEQ ID NO:2. In some embodiments, the antibody comprises (a) a heavy chain variable region (VH) comprising an amino acid sequence that is at least about 95%, 96%, 97%, 98%, 99% or 100% identical to the amino acid sequence of SEQ ID NO:1, and (b) a light chain variable region (VL) comprising an amino acid sequence that is at least about 95%, 96%, 97%, 98%, 99% or 100% identical to the amino acid sequence of SEQ ID NO:2.

In a particular embodiment, the antibody comprises (a) a heavy chain variable region (VH) comprising the amino acid sequence of SEQ ID NO: 1, and (b) a light chain variable region (VL) comprising the amino acid sequence of SEQ ID NO: 2.

In some embodiments, the antibody is a humanized antibody. In one embodiment, the antibody is an immunoglobulin molecule comprising a human constant region, particularly an IgG class immunoglobulin molecule comprising a human CH1, CH2, CH3 and/or CL domain. Exemplary sequences of human constant domains are given in SEQ ID NOs 10 and 11 (human kappa and lambda CL domains, respectively) and SEQ ID NO: 12 (human IgG1 heavy chain constant domains CH1-CH2-CH3). In some embodiments, the antibody comprises a light chain constant region comprising the amino acid sequence of SEQ ID NO: 10 or SEQ ID NO: 11, particularly the amino acid sequence of SEQ ID NO: 10. In some embodiments, the antibody comprises a heavy chain constant region comprising an amino acid sequence that is at least about 95%, 96%, 97%, 98%, 99% or 100% identical to the amino acid sequence of SEQ ID NO: 12. Particularly, the heavy chain constant region may comprise amino acid mutations in the Fc domain as described herein.

### Fc Domain

In particular embodiments, the antibody comprised in the immunconjugates for use according to the invention comprises an Fc domain, composed of a first and a second subunit. The Fc domain of an antibody consists of a pair of polypeptide chains comprising heavy chain domains of an immunoglobulin molecule. For example, the Fc domain of an immunoglobulin G (lgG) molecule is a dimer, each subunit of which comprises the CH2 and CH3 IgG heavy chain constant domains. The two subunits of the Fc domain are capable of stable association with each other. In one embodiment the immunoconjugate of the invention comprises not more than one Fc domain. In one embodiment the Fc domain of the antibody comprised in the immunoconjugate is an IgG Fc domain. In a particular embodiment the Fc domain is an IgG₁ Fc domain. In another embodiment the Fc domain is an IgG₄ Fc domain. In a more specific embodiment, the Fc domain is an IgG4 Fc domain comprising an amino acid substitution at position S228 (Kabat EU index numbering), particularly the amino acid substitution S228P. This amino acid substitution reduces in vivo Fab arm exchange of IgG4 antibodies (see Stubenrauch et al., Drug Metabolism and Disposition 38, 84-91 (2010)). In a further particular embodiment the Fc domain is a human Fc domain. In an even more particular embodiment, the Fc domain is a human IgG1 Fc domain. An exemplary sequence of a human IgG1 Fc region is given in SEQ ID NO: 9.

### Fc Domain Modifications Promoting Heterodimerization

Immunoconjugates for use according to the invention comprise a mutant IL-7 polypeptide, particularly a single (not more than one) mutant IL-7 polypeptide, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the immunoconjugate in recombinant production, it will thus be advantageous to introduce in the Fc domain of the antibody a modification promoting the association of the desired polypeptides.

Accordingly, in particular embodiments, the Fc domain of the antibody comprised in the immunoconjugate for use according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed).

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936: Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the antibody comprised in the immunoconjugate an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain (the “knobs” subunit) the threonine residue at position 366 is replaced with a tryptophan residue (T366W). and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter. J Immunol Methods 248, 7-15 (2001)).

In a particular embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index). In some embodiments, the second subunit of the Fc domain additionally comprises the amino acid substitutions H435R and Y436F (numbering according to Kabat EU index).

In a particular embodiment the mutant IL-7 polypeptide is fused (optionally through a linker peptide) to the first subunit of the Fc domain (comprising the “knob” modification). Without wishing to be bound by theory, fusion of the mutant IL-7 polypeptide to the knob-containing subunit of the Fc domain will (further) minimize the generation of immunoconjugates comprising two mutant IL-7 polypeptides (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment the heterodimerization approach described in EP 1870459, is used alternatively. This approach is based on the introduction of charged amino acids with opposite charges at specific amino acid positions in the CH3/CH3 domain interface between the two subunits of the Fc domain. One preferred embodiment for the antibody comprised in the immunoconjugates for use according to theinvention are amino acid mutations R409D; K370E in one of the two CH3 domains (of the Fc domain) and amino acid mutations D399K; E357K in the other one of the CH3 domains of the Fc domain (numbering according to Kabat EU index).

In another embodiment, the antibody comprised in the immunoconjugate for use according to the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another embodiment, the antibody comprised in the immunoconjugate for use according to the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment, the heterodimerization approach described in WO 2013/157953 is used alternatively. In one embodiment, a first CH3 domain comprises amino acid mutation T366K and a second CH3 domain comprises amino acid mutation L351D (numberings according to Kabat EU index). In a further embodiment, the first CH3 domain comprises further amino acid mutation L351K. In a further embodiment, the second CH3 domain comprises further an amino acid mutation selected from Y349E, Y349D and L368E (preferably L368E) (numberings according to Kabat EU index).

In one embodiment, the heterodimerization approach described in WO 2012/058768 is used alternatively. In one embodiment, a first CH3 domain comprises amino acid mutations L351Y, Y407A and a second CH3 domain comprises amino acid mutations T366A, K409F. In a further embodiment, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index).

In a further embodiment, a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment, the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A. In one embodiment, a first CH3 domain comprises amino acid mutation T366Y and a second CH3 domain comprises amino acid mutation Y407T (numberings according to Kabat EU index).

In one embodiment, the antibody comprised in the immunoconjugate or its Fc domain is of IgG2 subclass and the heterodimerization approach described in WO 2010/129304 is used alternatively.

In an alternative embodiment, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment, the first CH3 domain further comprises amino acid substitution of K409 or R409 with a negatively charged amino acid (e.g. glutamic acid (E), or aspartic acid (D), preferably K409D or R409D). In a further embodiment, the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment, the heterodimerization approach described in WO 2007/147901 is used alternatively. In one embodiment, a first CH3 domain comprises amino acid mutations K253E, D282K, and K322D and a second CH3 domain comprises amino acid mutations D239K, E240K, and K292D (numberings according to Kabat EU index).

In still another embodiment, the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the immunoconjugate favorable pharmacokinetic properties, including a long serum half-life which contributes to good accumulation in the target tissue and a favorable tissue-blood distribution ratio. At the same time it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-7 polypeptide and the long half-life of the immunoconjugate, results in excessive activation of cytokine receptors and severe side effects upon systemic administration. Accordingly, in particular embodiments, the Fc domain of the antibody comprised in the immunoconjugate for use according to the invention exhibits reduced binding affinity to an Fc receptor and/or reduced effector function, as compared to a native IgG₁ Fc domain. In one such embodiment the Fc domain (or the antibody comprising said Fc domain) exhibits less than 50%, preferably less than 20%, more preferably less than 10% and most preferably less than 5% of the binding affinity to an Fc receptor, as compared to a native IgG₁ Fc domain (or an antibody comprising a native IgG1 Fc domain), and/or less than 50%, preferably less than 20%, more preferably less than 10% and most preferably less than 5% of the effector function, as compared to a native IgGi Fc domain domain (or an antibody comprising a native IgG₁ Fc domain). In one embodiment, the Fc domain domain (or an antibody comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or an antibody comprising said Fc domain) exhibits greater than about 70%. particularly greater than about 80%, more particularly greater than about 90% of the binding affinity of a native IgG1 Fc domain (or an antibody comprising a native IgGi Fc domain) to FcRn. In certain embodiments the Fc domain is engineered to have reduced binding affinity to an Fc receptor and/or reduced effector function, as compared to a non-engineered Fc domain. In particular embodiments, the Fc domain of the antibody comprised in the immunoconjugate comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the antibody comprising an engineered Fc domain exhibits less than 20%, particularly less than 10%, more particularly less than 5% of the binding affinity to an Fc receptor as compared to an antibody comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to Clq, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or an antibody comprising said Fc domain) exhibits greater than about 70% of the binding affinity of a non-engineered form of the Fc domain (or an antibody comprising said non-engineered form of the Fc domain) to FcRn. The Fc domain, or antibody comprised in the immunoconjugate for use according to the invention comprising said Fc domain, may exhibit greater than about 80% and even greater than about 90% of such affinity. In certain embodiments the Fc domain of the antibody comprised in the immunoconjugate is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or an antibody comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329 (numberings according to Kabat EU index). In a more specific embodiment the Fc domain comprises an amino acid substitution at a position selected from the group of L234, L235 and P329 (numberings according to Kabat EU index). In some embodiments the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G (numberings according to Kabat EU index). In one embodiment the Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the Fc domain comprises amino acid substitutions at positions P329, L234 and L235 (numberings according to Kabat EU index). In more particular embodiments the Fc domain comprises the amino acid mutations L234A, L235A and P329G (“P329G LALA”, “PGLALA” or “LALAPG”). Specifically, in particular embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the antibody comprised in the immunoconjugate for use according to the invention is an IgG₄ Fc domain, particularly a human IgG₄ Fc domain. In one embodiment the IgG₄ Fc domain comprises amino acid substitutions at position S228, specifically the amino acid substitution S228P (numberings according to Kabat EU index). To further reduce its binding affinity to an Fc receptor and/or its effector function, in one embodiment the IgG₄ Fc domain comprises an amino acid substitution at position L235, specifically the amino acid substitution L235E (numberings according to Kabat EU index). In another embodiment, the IgG₄ Fc domain comprises an amino acid substitution at position P329, specifically the amino acid substitution P329G (numberings according to Kabat EU index). In a particular embodiment, the IgG₄ Fc domain comprises amino acid substitutions at positions S228, L235 and P329, specifically amino acid substitutions S228P, L235E and P329G (numberings according to Kabat EU index). Such IgG4 Fc domain mutants and their Fcγ receptor binding properties are described in PCT publication no. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), 20) and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing FcγIIIa receptor.

Effector function of an Fc domain, or an antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985): U.S. Pat. No. 5,821,337: Bruggemann et al., J Exp Med 166, 1351-1361 (1987). Alternatively, non-radioactive assays methods may be employed (see, for example, ACTIIM non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to Clq, is reduced. Accordingly, in some embodiments wherein the Fc domain is engineered to have reduced effector function, said reduced effector function includes reduced CDC. Clq binding assays may be carried out to determine whether the Fc domain, or antibody comprising the Fc domain, is able to bind Clq and hence has CDC activity. See e.g., Clq and C3c binding ELISA in WO 2006/029879 and WO 2005/100402. To assess complement activation, a CDC assay may be performed (see, for example, Gazzano-Santoro et al., J Immunol Methods 202, 163 (1996): Cragg et al., Blood 101, 1045-1052 (2003): and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18(12): 1759-1769 (2006): WO 2013/120929).

In one embodiment according to any of the above aspects of the invention, the antibody is an IgG class immunoglobulin, comprising a human IgG1 Fc domain composed of a first and a second subunit,

wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-7 polypeptide may be fused at its amino-terminal amino acid to the carboxy-terminal amino acid of the first subunit of the Fc domain, through a linker peptide of SEQ ID NO: 5.

In one aspect, the invention provides an immunoconjugate for use in the treatment of cancer comprising a polypeptide comprising an amino acid sequence that is at least about 80%, 85%. 90%, 95%, 96%, 97%, 98%, 99% or 100% identical to the sequence of SEQ ID NO:17, a polypeptide comprising an amino acid sequence that is at least about 80%, 85%, 90%, 95%, 96%, 97%, 98%, 99% or 100% identical to the sequence of SEQ ID NO:19, and a polypeptide comprising an amino acid sequence that is at least about 80%, 85%, 90%, 95%, 96%, 97%, 98%, 99% or 100% identical to the sequence of SEQ ID NO:24.

In one aspect, the invention provides an immunoconjugate for use in the treatment of cancer comprising a polypeptide comprising an amino acid sequence that is at least about 80%, 85%, 90%, 95%, 96%, 97%, 98%, 99% or 100% identical to the sequence of SEQ ID NO:17, a polypeptide comprising an amino acid sequence that is at least about 80%, 85%, 90%, 95%, 96%, 97%, 98%, 99% or 100% identical to the sequence of SEQ ID NO:19, and a polypeptide comprising an amino acid sequence that is at least about 80%, 85%, 90%, 95%, 96%, 97%, 98%, 99% or 100% identical to the sequence of SEQ ID NO:25.

In one aspect, the invention provides the use of an immunoconjugate in the manufacture of a medicament for the treatment of cancer comprising a polypeptide comprising an amino acid sequence that is at least about 80%, 85%, 90%, 95%, 96%, 97%, 98%, 99% or 100% identical to the sequence of SEQ ID NO:17, a polypeptide comprising an amino acid sequence that is at least about 80%, 85%, 90%, 95%, 96%, 97%, 98%, 99% or 100% identical to the sequence of SEQ ID NO:19, and a polypeptide comprising an amino acid sequence that is at least about 80%, 85%, 90%, 95%, 96%, 97%, 98%, 99% or 100% identical to the sequence of SEQ ID NO:24.

In one aspect, the invention provides the use of an immunoconjugate in the manufacture of a medicament for the treatment of cancer comprising a polypeptide comprising an amino acid sequence that is at least about 80%, 85%, 90%, 95%, 96%, 97%, 98%, 99% or 100% identical to the sequence of SEQ ID NO: 17, a polypeptide comprising an amino acid sequence that is at least about 80%, 85%, 90%, 95%, 96%, 97%, 98%, 99% or 100% identical to the sequence of SEQ ID

NO:19, and a polypeptide comprising an amino acid sequence that is at least about 80%, 85%, 90%, 95%, 96%, 97%, 98%, 99% or 100% identical to the sequence of SEQ ID NO:25.

In one aspect, the invention provides a method of treating cancer in an individual comprising administering to said individual a therapeutically effective amount an immunoconjugate comprising a polypeptide comprising an amino acid sequence that is at least about 80%, 85%, 90%, 95%, 96%, 97%, 98%, 99% or 100% identical to the sequence of SEQ ID NO:17, a polypeptide comprising an amino acid sequence that is at least about 80%, 85%, 90%, 95%, 96%, 97%, 98%, 99% or 100% identical to the sequence of SEQ ID NO:19, and a polypeptide comprising an amino acid sequence that is at least about 80%, 85%, 90%, 95%, 96%, 97%, 98%, 99% or 100% identical to the sequence of SEQ ID NO:24.

In one aspect, the invention provides a method of treating cancer in an individual comprising administering to said individual a therapeutically effective amount an immunoconjugate comprising a polypeptide comprising an amino acid sequence that is at least about 80%, 85%. 90%, 95%, 96%, 97%, 98%, 99% or 100% identical to the sequence of SEQ ID NO:17, a polypeptide comprising an amino acid sequence that is at least about 80%, 85%, 90%, 95%, 96%, 97%, 98%, 99% or 100% identical to the sequence of SEQ ID NO:19, and a polypeptide comprising an amino acid sequence that is at least about 80%, 85%, 90%, 95%, 96%, 97%, 98%, 99% or 100% identical to the sequence of SEQ ID NO:25.

### Recombinant Methods

Mutant IL-7 polypeptides useful in the invention can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. The sequence of native human IL-7 is shown in SEQ ID NO: 14. Substitution or insertion may involve natural as well as non-natural amino acid residues. Amino acid modification includes well known methods of chemical modification such as the addition of glycosylation sites or carbohydrate attachments, and the like.

Immunoconjugates for use according to the invention may be obtained, for example, by solid-state peptide synthesis (e.g. Merrifield solid phase synthesis) or recombinant production. For recombinant production one or more polynucleotide encoding the immunoconjugate (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides for use according to the invention is provided. Methods which are well known to those skilled in the art can be used to construct expression vectors containing the coding sequence of an immunoconjugate (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989): and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the immunoconjugate (fragment) (i.e. the coding region) is cloned in operable association with a promoter and/or other transcription or translation control elements.

As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid may encode heterologous coding regions, either fused or unfused to a polynucleotide encoding the immunoconjugates for use according to the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions may be associated with additional coding regions which encode secretory or signal peptides, which direct the secretion of a polypeptide encoded by a polynucleotide. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the immunoconjugate may be included within or at the ends of the immunoconjugate (fragment) encoding polynucleotide.

In a further embodiment, a host cell comprising one or more polynucleotides for use according to the invention is provided. The polynucleotides and vectors may incorporate any of the features, singly or in combination, described herein in relation to polynucleotides and vectors, respectively. In one such embodiment a host cell comprises (e.g. has been transformed or transfected with) one or more vector comprising one or more polynucleotide that encodes the immunoconjugate for use according to the invention. As used herein, the term “host cell” refers to any kind of cellular system which can be engineered to generate the immunoconjugates of the invention or fragments thereof. Host cells suitable for replicating and for supporting expression of immunoconjugates are well known in the art. Such cells may be transfected or transduced as appropriate with the particular expression vector and large quantities of vector containing cells can be grown for seeding large scale fermenters to obtain sufficient quantities of the immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293T cells as described, e.g., in Graham et al., J Gen Virol 36. 59 (1977)), baby hamster kidney cells (BHK), mouse sertoli cells (TM4 cells as described, e.g., in Mather, Biol Reprod 23, 243-251 (1980)), monkey kidney cells (CV1). African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few; but also cells comprised within a transgenic animal, transgenic plant or cultured plant or animal tissue.

Standard technologies are known in the art to express foreign genes in these systems. Cells expressing a mutant-IL-7 polypeptide fused to either the heavy or the light chain of an antibody may be engineered so as to also express the other of the antibody chains such that the expressed mutant IL-7 fusion product is an antibody that has both a heavy and a light chain.

In one embodiment, a method of producing an immunoconjugate for use according to the invention is provided, wherein the method comprises culturing a host cell comprising one or more polynucleotide encoding the immunoconjugate, as provided herein, under conditions suitable for expression of the immunoconjugate, and optionally recovering the immunoconjugate from the host cell (or host cell culture medium).

In the immunoconjugate for use according to the invention, the mutant IL-7 polypeptide may be genetically fused to the antibody, or may be chemically conjugated to the antibody. Genetic fusion of the IL-7 polypeptide to the antibody can be designed such that the IL-7 sequence is fused directly to the polypeptide or indirectly through a linker sequence. The composition and length of the linker may be determined in accordance with methods well known in the art and may be tested for efficacy. Particular linker peptides are described herein. Additional sequences may also be included to incorporate a cleavage site to separate the individual components of the fusion if desired, for example an endopeptidase recognition sequence. In addition, an IL-7 fusion protein may also be synthesized chemically using methods of polypeptide synthesis as is well known in the art (e.g. Merrifield solid phase synthesis). Mutant IL-7 polypeptides may be chemically conjugated to other molecules, e.g. antibodies, using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-7 and can readily be identified by those skilled in the art. Alternatively, or in addition, mutant IL-7 and/or the molecule to which it is intended to be conjugated may be chemically derivatized such that the two can be conjugated in a separate reaction as is also well known in the art.

The immunoconjugates for use according to the invention comprise an antibody. Methods to produce antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Immunoconjugates, antibodies, and methods for producing the same are also described in detail e.g. in PCT publication nos. WO 2011/020783, WO 2012/107417, and WO 2012/146628, each of which are incorporated herein by reference in their entirety.

Any animal species of antibody may be used in the immunoconjugates for use according to the invention. Non-limiting antibodies useful in the present invention can be of murine, primate, or human origin. If the immunoconjugate is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs: the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front. Biosci. 13:1619-1633 (2008), and are further described, e.g., in Riechmann et al., Nature 332:323-329 (1988): Queen et al., Proc. Natl Acad. Sci. USA 86:10029-10033 (1989): U.S. Pat. Nos. 5,821,337, 7,527,791, 6,982,321, and 7,087,409; Kashmiri et al., Methods 36:25-34 (2005) (describing specificity determining region (SDR) grafting): Padlan, Mol. Immunol. 28:489-498 (1991) (describing “resurfacing”): Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling). Human framework regions that may be used for humanization include but are not limited to: framework regions selected using the “best-fit” method (see, e.g., Sims et al. J. Immunol. 151:2296 (1993)): framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)): human mature (somatically mutated) framework regions or human germline framework regions (see, e.g., Almagro and Fransson, Front. Biosci. 13:1619-1633 (2008)): and framework regions derived from screening FR libraries (see, e.g., Baca et al., J. Biol. Chem. 272: 10678-10684 (1997) and Rosok et al., J. Biol. Chem. 271:22611-22618 (1996)).

Human antibodies can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology: U.S. Pat. No. 5,770,429 describing HUMAB® technology: U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984): Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3): 927-937 (2005) and Vollmers and Brandlein, Methods and Findings in Experimental and Clinical Pharmacology, 27(3): 185-91 (2005).

Human antibodies may also be generated by isolation from human antibody libraries, as described herein.

Antibodies useful in the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016): Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373: 7,985,840; 7,785,903 and 8,679,490 as well as US Patent Publication Nos. 2005/0079574, 2007/0117126, 2007/0237764 and 2007/0292936. Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Further chemical modification of the immunoconjugate for use in the invention may be desirable. For example, problems of immunogenicity and short half-life may be improved by conjugation to substantially straight chain polymers such as polyethylene glycol (PEG) or polypropylene glycol (PPG) (see e.g. WO 87/00056).

Immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immunoconjugate binds. For example, an antibody which specifically binds the mutant IL-7 polypeptide may be used. For affinity chromatography purification of immunoconjugates of the invention, a matrix with protein A or protein G may be used. For example, sequential Protein A or G affinity chromatography and size exclusion chromatography can be used to isolate an immunoconjugate essentially as described in the Examples. The purity of the immunoconjugate can be determined by any of a variety of well known analytical methods including gel electrophoresis, high pressure liquid chromatography, and the like.

### Compositions, Formulations, and Routes of Administration

In a further aspect, the invention provides pharmaceutical compositions comprising an immunoconjugate for use as described herein, e.g., for use in any of the below therapeutic methods. In one embodiment, a pharmaceutical composition comprises any of the immunoconjugates for the use provided herein and a pharmaceutically acceptable carrier. In another embodiment, a pharmaceutical composition comprises any of the immunoconjugates for the uses provided herein and at least one additional therapeutic agent, e.g., as described below.

Further provided is a method of producing an immunoconjugate for use according to the invention in a form suitable for administration in vivo, the method comprising (a) obtaining an immunoconjugate, and (b) formulating the immunoconjugate with at least one pharmaceutically acceptable carrier, whereby a preparation of immunoconjugate is formulated for administration in vivo.

Pharmaceutical compositions comprising an immunoconjugates for use according to the present invention comprise a therapeutically effective amount of immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

An immunoconjugate for use according to the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the immunoconjugates may be in powder form for constitution with a suitable vehicle, e.g., sterile pyrogen-free water, before use. Sterile injectable solutions are prepared by incorporating the immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginin, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the immunoconjugates for use according to the invention may also be formulated as a depot preparation. Such long acting formulations may be administered by implantation (for example subcutaneously or intramuscularly) or by intramuscular injection. Thus, for example, the immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the immunoconjugates for use according to the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides: or such organic bases as isopropylamine, trimethylamine, histidine or procaine. Pharmaceutical salts tend to be more soluble in aqueous and other protic solvents than are the corresponding free base forms.

### Therapeutic Methods

The immunoconjugates provided herein may be used in therapeutic methods and uses. Immunoconjugates may be used as immunotherapeutic agents, for example in the treatment of cancers.

For use in therapeutic methods according to the invention, immunoconjugates would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Immunoconjugates for use according to the invention may be particularly useful in treating disease states where stimulation of the immune system of the host is beneficial, in particular conditions where an enhanced cellular immune response is desirable. These may include disease states where the host immune response is insufficient or deficient. Disease states for which the immunoconjugates may be administered comprise, for example, a tumor or infection where a cellular immune response would be a critical mechanism for specific immunity. The immunoconjugates for use according to the invention may be administered per se or in any suitable pharmaceutical composition.

In a further aspect, the invention provides a method for treating a disease in an individual. In one embodiment, the method comprises administering to an individual having such disease a therapeutically effective amount of an immunoconjugate. In one embodiment a composition is administered to said invididual, comprising the immunoconjugate in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of an immunoconjugate to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of kidney cancer, skin cancer, lung cancer, colorectal cancer, breast cancer, brain cancer, head and neck cancer, prostate cancer and bladder cancer. A skilled artisan readily recognizes that in many cases the immunoconjugates may not provide a cure but may only provide partial benefit. In some embodiments, a physiological change having some benefit is also considered therapeutically beneficial. Thus, in some embodiments, an amount of immunoconjugate that provides a physiological change is considered an “effective amount” or a “therapeutically effective amount”. The subject, patient, or individual in need of treatment is typically a mammal, more specifically a human.

In some embodiments, an effective amount of an immunoconjugate is administered to a cell. In other embodiments, a therapeutically effective amount of an immunoconjugates is administered to an individual for the treatment of disease.

For the prevention or treatment of disease, the appropriate dosage of an immunoconjugate of the invention (when used alone or in combination with one or more other additional therapeutic agents) will depend on the type of disease to be treated, the route of administration, the body weight of the patient, the type of molecule (e.g. comprising an Fc domain or not), the severity and course of the disease, whether the immunoconjugate is administered for preventive or therapeutic purposes, previous or concurrent therapeutic interventions, the patient's clinical history and response to the immunoconjugate, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The immunoconjugate is suitably administered to the patient at one time or over a series of treatments. Depending on the type and severity of the disease, about 1 μg/kg to 15 mg/kg (e.g. 0.1 mg/kg-10 mg/kg) of immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the immunoconjugate would be in the range from about 0.005 mg/kg to about 10 mg/kg. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the immunoconjugate). An initial higher loading dose, followed by one or more lower doses may be administered. However, other dosage regimens may be useful. The progress of this therapy is easily monitored by conventional techniques and assays.

The immunoconjugates for use according to the invention will generally be used in an amount effective to achieve the intended purpose. For use to treat or prevent a disease condition, the immunoconjugates, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the ICso as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the immunoconjugates which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the immunoconjugates may not be related to plasma concentration. One having skill in the art will be able to optimize therapeutically effective local dosages without undue experimentation.

A therapeutically effective dose of the immunoconjugates described herein will generally provide therapeutic benefit without causing substantial toxicity. Toxicity and therapeutic efficacy of an immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Immunoconjugates that exhibit large therapeutic indices are preferred. In one embodiment, the immunoconjugate for use according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with immunoconjugates would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The maximum therapeutic dose of an immunoconjugate comprising a mutant IL-7 polypeptide as described herein may be increased from those used for an immunoconjugate comprising wild-type IL-7.

### Other Agents and Treatments

The immunoconjugates for use according to the invention may be administered in combination with one or more other agents in therapy. For instance, an immunoconjugate may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of immunoconjugate used, the type of disorder or treatment, and other factors discussed above. The immunoconjugates are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the immunoconjugate of the invention can occur prior to, simultaneously, and/or following, administration of the additional therapeutic agent and/or adjuvant. Immunoconjugates for use according to the invention may also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an immunoconjugate of the invention. The label or package insert indicates that the composition is used for treating the condition of choice. Moreover, the article of manufacture may comprise (a) a first container with a composition contained therein, wherein the composition comprises an immunoconjugate for use according to the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Amino Acid Sequences

| | | SEQ ID |
|---|---|---|
| | Amino Acid Sequence | NO |
| PD1(alt) VH | QVQLQQPGAELVKPGTSVKLSCKASGYTFTNYYMYWVK | 1 |
| | QRPGQGLEWIGGINPSNGGTNFNEKFKNKATLTVDSSSST | |
| | TYMQLSSLTSEDSAVYYCTRRDYRFDMGFDYWGQGTTL | |
| | TVSS | |
| PD1(alt) VL | DIVLTQSPASLAVSLGQRAAISCRASKGVSTSGYSYLHWY | 2 |
| | QQKPGQSPKLLIYLASYLESGVPARFSGSGSGTDFTLNIHP | |
| | VEEEDAATYYCQHSRDLPLTFGTGTKLELK | |
| PD1 VH | EVQLLESGGGLVQPGGSLRLSCAASGFSFSSYTMSWVRQ | 3 |
| | APGKGLEWVATISGGGRDIYYPDSVKGRFTISRDNSKNTL | |
| | YLQMNSLRAEDTAVYYCVLLTGRVYFALDSWGQGTLVT | |
| | VSS | |
| PD-1 VL | DIVMTQSPDSLAVSLGERATINCKASESVDTSDNSFIHWY | 4 |
| | QQKPGQSPKLLIYRSSTLESGVPDRFSGSGSGTDFTLTISSL | |
| | QAEDVAVYYCQQNYDVPWTFGQGTKVEIK | |
| Linker | GGGGS GGGGS GGGG | 5 |
| Alternative | GGGGS GGGGS GGGGS | 6 |
| Linker | | |
| hPD-1 | PGWFLDSPDRPWNPPTFSPALLVVTEGDNATFTCSFSNTS | 7 |
| (without signal | ESFVLNWYRMSPSNQTDKLAAFPEDRSQPGQDCRFRVTQ | |
| sequence) | LPNGRDFHMSVVRARRNDSGTYLCGAISLAPKAQIKESLR | |
| | AELRVTERRAEVPTAHPSPSPRPAGQFQTLVVGVVGGLLG | |
| | SLVLLVWVLAVICSRAARGTIGARRTGQPLKEDPSAVPVF | |
| | SVDYGELDFQWREKTPEPPVPCVPEQTEYATIVFPSGMGT | |
| | SSPARRGSADGPRSAQPLRPEDGHCSWPL | |
| hPD-1 (with | MQIPQAPWPVVWAVLQLGWRPGWFLDSPDRPWNPPTFS | 8 |
| signal | PALLVVTEGDNATFTCSFSNTSESFVLNWYRMSPSNQTDK | |
| sequence) | LAAFPEDRSQPGQDCRFRVTQLPNGRDFHMSVVRARRND | |
| | SGTYLCGAISLAPKAQIKESLRAELRVTERRAEVPTAHPSP | |
| | SPRPAGQFQTLVVGVVGGLLGSLVLLVWVLAVICSRAAR | |
| | GTIGARRTGQPLKEDPSAVPVFSVDYGELDFQWREKTPEP | |
| | PVPCVPEQTEYATIVFPSGMGTSSPARRGSADGPRSAQPL | |
| | RPEDGHCSWPL | |
| Human IgG1 | DKTHTCPPCPAPELLGGPSVFLFPPKPKDTLMISRTPEVTC | 9 |
| Fc domain | VVVDVSHEDPEVKFNWYVDGVEVHNAKTKPREEQYNST | |
| | YRVVSVLTVLHQDWLNGKEYKCKVSNKALPAPIEKTISK | |
| | AKGQPREPQVYTLPPSRDELTKNQVSLTCLVKGFYPSDIA | |
| | VEWESNGQPENNYKTTPPVLDSDGSFFLYSKLTVDKSRW | |
| | QQGNVFSCSVMHEALHNHYTQKSLSLSP | |
| Human kappa | RTVAAPSVFIFPPSDEQLKSGTASVVCLLNNFYPREAKVQ | 10 |
| CL domain | WKVDNALQSGNSQESVTEQDSKDSTYSLSSTLTLSKADY | |
| | EKHKVYACEVTHQGLSSPVTKSFNRGEC | |
| Human | QPKAAPSVTLFPPSSEELQANKATLVCLISDFYPGAVTVA | 11 |
| lambda CL | WKADSSPVKAGVETTTPSKQSNNKYAASSYLSLTPEQWK | |
| domain | SHRSYSCQVTHEGSTVEKTVAPTECS | |
| Human IgG1 | ASTKGPSVFPLAPSSKSTSGGTAALGCLVKDYFPEPVTVS | 12 |
| heavy chain | WNSGALTSGVHTFPAVLQSSGLYSLSSVVTVPSSSLGTQT | |
| constant | YICNVNHKPSNTKVDKKVEPKSCDKTHTCPPCPAPELLGG | |
| region (CH1- | PSVFLFPPKPKDTLMISRTPEVTCVVVDVSHEDPEVKFNW | |
| CH2-CH3) | YVDGVEVHNAKTKPREEQYNSTYRVVSVLTVLHQDWLN | |
| | GKEYKCKVSNKALPAPIEKTISKAKGQPREPQVYTLPPSR | |
| | DELTKNQVSLTCLVKGFYPSDIAVEWESNGQPENNYKTT | |
| | PPVLDSDGSFFLYSKLTVDKSRWQQGNVFSCSVMHEALH | |
| | NHYTQKSLSLSP | |
| hPD-1 | PGWFLDSPDRPWNPPTFSPALLVVTEGDNATFTCSFSNTS | 13 |
| Extracellular | ESFVLNWYRMSPSNQTDKLAAFPEDRSQPGQDCRFRVTQ | |
| Domain | LPNGRDFHMSVVRARRNDSGTYLCGAISLAPKAQIKESLR | |
| (ECD) | AELRVTERRAEVPTAHPSPSPRPAGQFQTLV | |

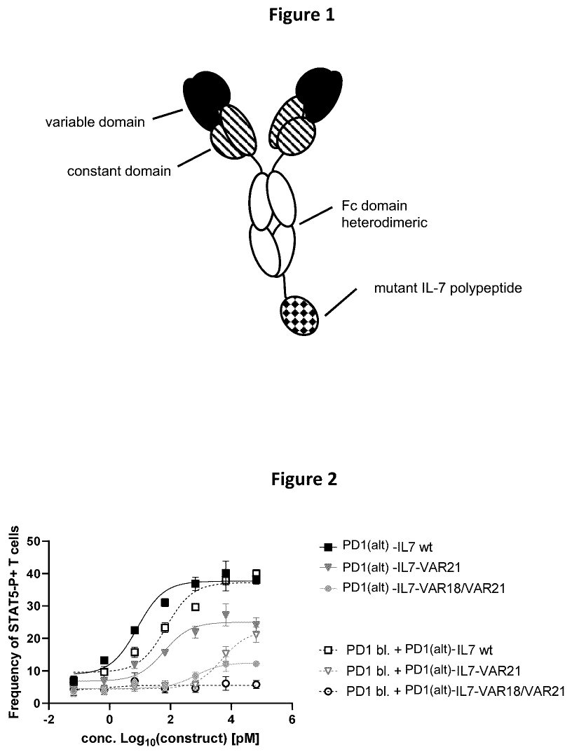

Amino acid sequences re IL-7

| | | | SEQ ID |
|---|---|---|---|
| | Modification | Amino acid sequece | NO |
| Human IL7 | — | DCDIEGKDGKQYESVLMVSIDQLLDSMKEIG | 14 |
| wild type | | SNCLNNEFNFFKRHICDANKEGMFLFRAAR | |
| | | KLRQFLKMNSTGDFDLHLLKVSEGTTILLNC | |
| | | TGQVKGRKPAALGEAQPTKSLEENKSLKEQ | |
| | | KKLNDLCFLKRLLQEIKTCWNKILMGTKEH | |
| IL7-VAR21 | G85E | DCDIEGKDGKQYESVLMVSIDQLLDSMKEIG | 15 |
| | | SNCLNNEFNFFKRHICDANKEGMFLFRAAR | |
| | | KLRQFLKMNSTGDFDLHLLKVSEETTILLNC | |
| | | TGQVKGRKPAALGEAQPTKSLEENKSLKEQ | |
| | | KKLNDLCFLKRLLQEIKTCWNKILMGTKEH | |
| IL7- | K81E, G85E | DCDIEGKDGKQYESVLMVSIDQLLDSMKEIG | 16 |
| VAR18/VAR21 | | SNCLNNEFNFFKRHICDANKEGMFLFRAAR | |
| | | KLRQFLKMNSTGDFDLHLLEVSEETTILLNC | |
| | | TGQVKGRKPAALGEAQPTKSLEENKSLKEQ | |
| | | KKLNDLCFLKRLLQEIKTCWNKILMGTKEH | |
| Chain A | | DIVLTQSPASLAVSLGQRAAISCRASKGVSTS | 17 |
| PD1(alt) | | GYSYLHWYQQKPGQSPKLLIYLASYLESGV | |
| | | PARFSGSGSGTDFTLNIHPVEEEDAATYYCQ | |
| | | HSRDLPLTFGTGTKLELKRTVAAPSVFIFPPS | |
| | | DEQLKSGTASVVCLLNNFYPREAKVQWKV | |
| | | DNALQSGNSQESVTEQDSKDSTYSLSSTLTL | |
| | | SKADYEKHKVYACEVTHQGLSSPVTKSFNR | |
| | | GEC | |
| Chain A PD1 | — | DIVMTQSPDSLAVSLGERATINCKASESVDT | 18 |
| | | SDNSFIHWYQQKPGQSPKLLIYRSSTLESGVP | |
| | | DRFSGSGSGTDFTLTISSLQAEDVAVYYCQQ | |
| | | NYDVPWTFGQGTKVEIKRTVAAPSVFIFPPS | |
| | | DEQLKSGTASVVCLLNNFYPREAKVQWKV | |
| | | DNALQSGNSQESVTEQDSKDSTYSLSSTLTL | |
| | | SKADYEKHKVYACEVTHQGLSSPVTKSFNR | |
| | | GEC | |
| Chain H | | QVQLQQPGAELVKPGTSVKLSCKASGYTFT | 19 |
| PD1(alt) | | NYYMYWVKQRPGQGLEWIGGINPSNGGTN | |
| C2184220335 | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVCTLPPSRDELTK | |
| | | NQVSLSCAVKGFYPSDIAVEWESNGQPENN | |
| | | YKTTPPVLDSDGSFFLVSKLTVDKSRWQQG | |
| | | NVFSCSVMHEALHNRFTQKSLSLSPG | |
| Chain H | | EVQLLESGGGLVQPGGSLRLSCAASGFSFSS | 20 |
| (PD1-IL7wt, | | YTMSWVRQAPGKGLEWVATISGGGRDIYYP | |
| Ref mol 5-8) | | DSVKGRFTISRDNSKNTLYLQMNSLRAEDT | |
| C01483382 | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVCTLPPSRDELTKNQV | |
| | | SLSCAVKGFYPSDIAVEWESNGQPENNYKTT | |
| | | PPVLDSDGSFFLVSKLTVDKSRWQQGNVFS | |
| | | CSVMHEALHNHYTQKSLSLSPGK | |
| Chain H | — | EVQLLESGGGLVQPGGSLRLSCAASGFSFSS | 21 |
| (Ref mol 9 + | | YTMSWVRQAPGKGLEWVATISGGGRDIYYP | |
| 10) | | DSVKGRFTISRDNSKNTLYLQMNSLRAEDT | |
| C2184230856 | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVCTLPPSRDELTKNQV | |
| | | SLSCAVKGFYPSDIAVEWESNGQPENNYKTT | |
| | | PPVLDSDGSFFLVSKLTVDKSRWQQGNVFS | |
| | | CSVMHEALHNHYTQKSLSLSPGK | |
| Chain K of | | QVQLQQPGAELVKPGTSVKLSCKASGYTFT | 22 |
| PD1(alt)-IL7wt | | NYYMYWVKQRPGQGLEWIGGINPSNGGTN | |
| | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVYTLPPCRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGGGGG | |
| | | SGGGGSGGGGDCDIEGKDGKQYESVLMVSI | |
| | | DQLLDSMKEIGSNCLNNEFNFFKRHICDANK | |
| | | EGMFLFRAARKLRQFLKMNSTGDFDLHLLK | |
| | | VSEGTTILLNCTGQVKGRKPAALGEAQPTKS | |
| | | LEENKSLKEQKKLNDLCFLKRLLQEIKTCW | |
| | | NKILMGTKEH | |
| Chain K of | — | EVQLLESGGGLVQPGGSLRLSCAASGFSFSS | 23 |
| PD1-IL7wt | | YTMSWVRQAPGKGLEWVATISGGGRDIYYP | |
| | | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGGSGGG | |
| | | GSGGGGSDCDIEGKDGKQYESVLMVSIDQL | |
| | | LDSMKEIGSNCLNNEFNFFKRHICDANKEG | |
| | | MFLFRAARKLRQFLKMNSTGDFDLHLLKVS | |
| | | EGTTILLNCTGQVKGRKPAALGEAQPTKSLE | |
| | | ENKSLKEQKKLNDLCFLKRLLQEIKTCWNKI | |
| | | LMGTKEH | |
| Chain K of | G85E | QVQLQQPGAELVKPGTSVKLSCKASGYTFT | 24 |
| PD1(alt)-IL7 | | NYYMYWVKQRPGQGLEWIGGINPSNGGTN | |
| VAR21 | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVYTLPPCRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGGGGG | |
| | | SGGGGSGGGGDCDIEGKDGKQYESVLMVSI | |
| | | DQLLDSMKEIGSNCLNNEFNFFKRHICDANK | |
| | | EGMFLFRAARKLRQFLKMNSTGDFDLHLLK | |
| | | VSEETTILLNCTGQVKGRKPAALGEAQPTKS | |
| | | LEENKSLKEQKKLNDLCFLKRLLQEIKTCW | |
| | | NKILMGTKEH | |
| Chain K of | K81E, G85E | QVQLQQPGAELVKPGTSVKLSCKASGYTFT | 25 |
| PD1(alt)-IL7 | | NYYMYWVKQRPGQGLEWIGGINPSNGGTN | |
| VAR18/VAR21 | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVYTLPPCRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGGGGG | |
| | | SGGGGSGGGGDCDIEGKDGKQYESVLMVSI | |
| | | DQLLDSMKEIGSNCLNNEFNFFKRHICDANK | |
| | | EGMFLFRAARKLRQFLKMNSTGDFDLHLLE | |
| | | VSEETTILLNCTGQVKGRKPAALGEAQPTKS | |
| | | LEENKSLKEQKKLNDLCFLKRLLQEIKTCW | |
| | | NKILMGTKEH | |
| Chain K of Ref | D74E | EVQLLESGGGLVQPGGSLRLSCAASGFSFSS | 26 |
| mol 5 | | YTMSWVRQAPGKGLEWVATISGGGRDIYYP | |
| | | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGGSGGG | |
| | | GSGGGGSDCDIEGKDGKQYESVLMVSIDQL | |
| | | LDSMKEIGSNCLNNEFNFFKRHICDANKEG | |
| | | MFLFRAARKLRQFLKMNSTGEFDLHLLKVS | |
| | | EGTTILLNCTGQVKGRKPAALGEAQPTKSLE | |
| | | ENKSLKEQKKLNDLCFLKRLLQEIKTCWNKI | |
| | | LMGTKEH | |
| Chain K of Ref | SS2 | EVQLLESGGGLVQPGGSLRLSCAASGFSFSS | 27 |
| mol 6 | (C2S/C141S, | YTMSWVRQAPGKGLEWVATISGGGRDIYYP | |
| | C47S/C92S) | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGGSGGG | |
| | | GSGGGGSDSDIEGKDGKQYESVLMVSIDQL | |
| | | LDSMKEIGSNCLNNEFNFFKRHISDANKEGM | |
| | | FLFRAARKLRQFLKMNSTGDFDLHLLKVSE | |
| | | GTTILLNSTGQVKGRKPAALGEAQPTKSLEE | |
| | | NKSLKEQKKLNDLCFLKRLLQEIKTSWNKIL | |
| | | MGTKEH | |
| Chain K of Ref | SS3 | EVQLLESGGGLVQPGGSLRLSCAASGFSFSS | 28 |
| mol 7 | (C47S/C92S, | YTMSWVRQAPGKGLEWVATISGGGRDIYYP | |
| | C34S/C129S) | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGGSGGG | |
| | | GSGGGGSDCDIEGKDGKQYESVLMVSIDQL | |
| | | LDSMKEIGSNSLNNEFNFFKRHISDANKEGM | |
| | | FLFRAARKLRQFLKMNSTGDFDLHLLKVSE | |
| | | GTTILLNSTGQVKGRKPAALGEAQPTKSLEE | |
| | | NKSLKEQKKLNDLSFLKRLLQEIKTCWNKIL | |
| | | MGTKEH | |
| Chain K of Ref | W142H | EVQLLESGGGLVQPGGSLRLSCAASGFSFSS | 29 |
| mol 8 | | YTMSWVRQAPGKGLEWVATISGGGRDIYYP | |
| | | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGGSGGG | |
| | | GSGGGGSDCDIEGKDGKQYESVLMVSIDQL | |
| | | LDSMKEIGSNCLNNEFNFFKRHICDANKEG | |
| | | MFLFRAARKLRQFLKMNSTGDFDLHLLKVS | |
| | | EGTTILLNCTGQVKGRKPAALGEAQPTKSLE | |
| | | ENKSLKEQKKLNDLCFLKRLLQEIKTCHNKI | |
| | | LMGTKEH | |
| Chain K of Ref | D74N | EVQLLESGGGLVQPGGSLRLSCAASGFSFSS | 30 |
| mol 9 | | YTMSWVRQAPGKGLEWVATISGGGRDIYYP | |
| | | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGSGGGG | |
| | | SGGGGDCDIEGKDGKQYESVLMVSIDQLLD | |
| | | SMKEIGSNCLNNEFNFFKRHICDANKEGMFL | |
| | | FRAARKLRQFLKMNSTGNFDLHLLKVSEGT | |
| | | TILLNCTGQVKGRKPAALGEAQPTKSLEENK | |
| | | SLKEQKKLNDLCFLKRLLQEIKTCWNKILM | |
| | | GTKEH | |
| Chain K of Ref | D74N, K81E | EVQLLESGGGLVQPGGSLRLSCAASGFSFSS | 31 |
| mol 10 | | YTMSWVRQAPGKGLEWVATISGGGRDIYYP | |
| | | DSVKGRFTISRDNSKNTLYLQMNSLRAEDT | |
| | | AVYYCVLLTGRVYFALDSWGQGTLVTVSSA | |
| | | STKGPSVFPLAPSSKSTSGGTAALGCLVKDY | |
| | | FPEPVTVSWNSGALTSGVHTFPAVLQSSGLY | |
| | | SLSSVVTVPSSSLGTQTYICNVNHKPSNTKV | |
| | | DKKVEPKSCDKTHTCPPCPAPEAAGGPSVFL | |
| | | FPPKPKDTLMISRTPEVTCVVVDVSHEDPEV | |
| | | KFNWYVDGVEVHNAKTKPREEQYNSTYRV | |
| | | VSVLTVLHQDWLNGKEYKCKVSNKALGAPI | |
| | | EKTISKAKGQPREPQVYTLPPCRDELTKNQV | |
| | | SLWCLVKGFYPSDIAVEWESNGQPENNYKT | |
| | | TPPVLDSDGSFFLYSKLTVDKSRWQQGNVFS | |
| | | CSVMHEALHNHYTQKSLSLSPGGGGSGGGG | |
| | | SGGGGDCDIEGKDGKQYESVLMVSIDQLLD | |
| | | SMKEIGSNCLNNEFNFFKRHICDANKEGMFL | |
| | | FRAARKLRQFLKMNSTGNFDLHLLEVSEGT | |
| | | TILLNCTGQVKGRKPAALGEAQPTKSLEENK | |
| | | SLKEQKKLNDLCFLKRLLQEIKTCWNKILM | |
| | | GTKEH | |
| Chain A-FIG.1 | | DIVMTQSPDSLAVSLGERATINCKASESVDT | 32 |
| | | SDNSFIHWYQQKPGQSPKLLIYRSSTLESGVP | |
| | | DRFSGSGSGTDFTLTISSLQAEDVAVYYCQQ | |
| | | NYDVPWTFGQGTKVEIKRTVAAPSVFIFPPS | |
| | | DEQLKSGTASVVCLLNNFYPREAKVQWKV | |
| | | DNALQSGNSQESVTEQDSKDSTYSLSSTLTL | |
| | | SKADYEKHKVYACEVTHQGLSSPVTKSFNR | |
| | | GEC | |
| Chain H-FIG.1 | | QVQLQQPGAELVKPGTSVKLSCKASGYTFT | 33 |
| | | NYYMYWVKQRPGQGLEWIGGINPSNGGTN | |
| | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVCTLPPSRDELTK | |
| | | NQVSLSCAVKGFYPSDIAVEWESNGQPENN | |
| | | YKTTPPVLDSDGSFFLVSKLTVDKSRWQQG | |
| | | NVFSCSVMHEALHNRFTQKSLSLSPG | |
| Chain K of | 34 | QVQLQQPGAELVKPGTSVKLSCKASGYTFT | |
| PD1(alt)-IL7-wt- | | NYYMYWVKQRPGQGLEWIGGINPSNGGTN | |
| FIG. 1 | | FNEKFKNKATLTVDSSSSTTYMQLSSLTSED | |
| | | SAVYYCTRRDYRFDMGFDYWGQGTTLTVS | |
| | | SASTKGPSVFPLAPSSKSTSGGTAALGCLVK | |
| | | DYFPEPVTVSWNSGALTSGVHTFPAVLQSSG | |
| | | LYSLSSVVTVPSSSLGTQTYICNVNHKPSNT | |
| | | KVDKKVEPKSCDKTHTCPPCPAPEAAGGPS | |
| | | VFLFPPKPKDTLMISRTPEVTCVVVDVSHED | |
| | | PEVKFNWYVDGVEVHNAKTKPREEQYNST | |
| | | YRVVSVLTVLHQDWLNGKEYKCKVSNKAL | |
| | | GAPIEKTISKAKGQPREPQVYTLPPCRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGGGGG | |
| | | SGGGGSGGGGDCDIEGKDGKQYESVLMVSI | |
| | | DQLLDSMKEIGSNCLNNEFNFFKRHICDANK | |
| | | EGMFLFRAARKLRQFLKMNSTGDFDLHLLK | |
| | | VSEGTTILLNCTGQVKGRKPAALGEAQPTKS | |
| | | LEENKSLKEQKKLNDLCFLKRLLQEIKTCW | |
| | | NKILMGTKEH | |

### EXAMPLES

The following are examples of methods and compositions of the invention. It is understood that various other embodiments may be practiced, given the general description provided above.

An exemplary format of an immunoconjugates according to the invention is shown as schematic representations in FIG. 1. The IgG-IL7 immunoconjugate comprises two Fab domains (variable domain, constant domain), a heterodimeric Fc domain and a mutant IL-7 polypeptide fused to a C-terminus of the Fc domain. The IgG-IL7 immunoconjugate is composed of polypeptides of amino acid sequences according to SEQ ID NO: 32, SEQ ID NO: 33 and SEQ ID NO: 34.

The sequences provided for the exemplary format relate to immunoconjugate with an IL-7 wild-type sequences. However, any mutant IL-7 polpypetide as disclosed herein may be incorporated in said formats instead of a wild-type IL-7.

### Example 1

### Example 1.1 Production and Analytics of PD1 Antibody IL7v Fusion Proteins

The antibody IL7 variant fusion constructs described in Table 1 were produced in CHO cells. The proteins were purified by ProteinA affinity chromatography and size exclusion chromatography. The end product analytics consists of monomer content determination (by analytical size exclusion chromatography) and percentage of main peak (determined by non-reduced capillary SDS electrophoresis: CE-SDS). Reference molecule 6 comprises an IL7 moiety as disclosed in WO 2020/127377 A1. Reference molecules 9 and 10 comprise IL7 moieties as disclosed in WO 2020/236655 A1. They are of the same format as other fusion constructs disclosed herein comprising one IL7 moiety fused to the N-terminal of the PD-1 antibody (FIG. 1). PD1(alt)-IL7wt, PD1(alt)-IL7 VAR21 and PD1(alt)-IL7 VAR 18/VAR21 comrpise a PD1 binder with a VH domain according to the sequence of SEQ ID NO: 1 and a VL domain according to the sequence of SEQ ID NO: 2. PD1-IL7wt and the reference molecules 5-10 comprise a PD1 binder with a VH domain according to the sequence of SEQ ID NO: 3 and a VL domain according to the sequence of SEQ ID NO: 4.

TABLE 1
Polypeptide amino acid sequences of
tested PD1-IL7 fusion proteins.

| Description | IL7 variant | ID | SEQ ID NOs |
|---|---|---|---|
| PD1-IL7wt | — | P1AF5572-018 | 18, 20, 23 |
| PD1(alt)-IL7wt | — | P1AG3437-001 | 17, 19, 22 |
| PD1(alt)-IL7-VAR21 | G85E | P1AG3439-001 | 17, 19, 24 |
| PD1(alt)-IL7- | K81E, G85E | P1AG3441-001 | 17, 19, 25 |
| VAR18/VAR21 | | | |
| Reference molecule 6 | SS2 (C2S/C141S, | P1AF9648-033 | 18, 20, 27 |
| | C47S/C92S) | | |
| Reference molecule 9 | D74N | P1AG8273-001 | 18, 21, 30 |
| Reference molecule 10 | D74N/K81E | P1AG8275-001 | 18, 21, 31 |

Cloning. Expression of all genes is under control of a human CMV promoter.

Production of IgG-like proteins in CHO K1 cells. The antibodies described herein were prepared by WuXi Biologics using their proprietary vector system with conventional (non-PCR based) cloning techniques and using suspension-adapted CHO KI cells. For the production, WuXi Biologics used commercially available chemically defined media and cultivated the cells after transfection under the following conditions: 36.5 C+6% Carbon Dioxide.

The supernatants were harvested by centrifugation and subsequent filtration (0.2 μm filter) and, proteins were purified from the harvested supernatant by standard methods.

Titer determination (PA-HPLC). Quantification of Fc containing constructs in supernatants was performed by Protein A-HPLC on an Agilent HPLC System with UV detector. Supernatants are injected on POROS 20 A (Applied Biosystems). The eluted peak area at 280 nm is integrated and converted to concentration by use of a calibration curve with standards analyzed in the same run.

Purification of IgG-like proteins. Proteins were purified from filtered cell culture supernatants referring to standard protocols. In brief, Fc containing proteins were purified from cell culture superatants by Protein A-affinity chromatography. Elution was followed by immediate pH neutralization of the sample. The protein was concentrated by centrifugation (Millipore Amicon® ULTRA-15; Art. Nr.: UFC903096), and aggregated protein was separated from monomeric protein by size exclusion chromatography (Äkta Pure & HiLoad 26/600 Superdex 200; both from Cytiva formally known as GE Healthcare) in 20 mM histidine, 140 mM sodium chloride, pH 6.0.

Analytics of IgG-like proteins. The concentrations of purified proteins were determined by measuring the absorption at 280 nm (Little Lunatic formally known as Dropsense 16; Unchained labs) using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25°C. using an analytical size-exclusion column (TSKgel G3000 SW XL).

TABLE 2
Monomer product peak, high molecular weight (HMW) and
low molecular weight (LMW) side products determined
by analytical size exclusion chromatography (SEC).

| | | Monomer | HMW | LMW |
|---|---|---|---|---|
| | | peak | peak | peak |
| PD1-IL7 variant | ID | (%) | (%) | (%) |
| PD1-IL7wt | P1AF5572-018 | 99.6 | 0.4 | 0 |
| PD1(alt)-IL7wt | P1AG3437-001 | 100 | 0 | 0 |
| PD1(alt)-IL7-VAR21 | P1AG3439-001 | 99.2 | 0.8 | 0 |
| PD1(alt)-IL7- | P1AG3441-001 | 99.5 | 0.5 | 0 |
| VAR18/VAR21 | | | | |
| Reference molecule 6 | P1AF9648-033 | 99.1 | 0.9 | 0 |
| Reference molecule 9 | P1AG8273-001 | 96.1 | 0.5 | 3.4 |
| Reference molecule 10 | P1AG8275-001 | 93.3 | 0.7 | 6 |

TABLE 3
Main product peak determined by non-reduced CE-SDS.

| PD1-IL7 variant | ID | Main peak (%) |
|---|---|---|
| PD1-IL7wt | P1AF5572-018 | 100 |
| PD1(alt)-IL7wt | P1AG3437-001 | 99.4 |
| PD1(alt)-IL7-VAR21 | P1AG3439-001 | 99.5 |
| PD1(alt)-IL7- | P1AG3441-001 | 99.3 |
| VAR18/VAR21 | | |
| Reference molecule 6 | P1AF9648-033 | 100 |
| Reference molecule 9 | P1AG8273-001 | 95.6 |
| Reference molecule 10 | P1AG8275-001 | 93.6 |

Results. The purified PD1-IL7 variant constructs were purified by ProteinA and size exclusion chromatography. The quality analysis of the purified material revealed that the monomer content was above 93% by analytical size exclusion chromatography analysis (Table 2) and that the main product peak was between 95% and 99% by non-reduced capillary electrophoresis (Table 3). In conclusion, all PD1-IL7 variants were produced in good quality.

### Example 1.2 Production and Analytics of Further PD1-IL7v Fusion Proteins (Reference Molecules 5, 7 and 8)

The antibody IL7 variants fusion constructs described in Table 4 were produced in CHO cells. The proteins were purified by ProteinA affinity chromatography and size exclusion chromatography. The end product analytics consists of monomer content determination (by analytical size exclusion chromatography) and percentage of main peak (determined by non-reduced capillary SDS electrophoresis: CE-SDS). Reference molecules 5, 7 and 8 comprise IL7 moieties as disclosed in WO 2020/127377 A1. They are of the same format as other fusion constructs disclosed herein comprising one IL7 moiety fused to the N-terminal of the PD-1 antibody (FIG. 1). Reference molecules 5, 7 and 8 comprise a PD1 binder with a VH domain according to the sequence of SEQ ID NO: 3 and a VL domain according to the sequence of SEQ ID NO: 4.

TABLE 4
Polypeptide amino acid sequences
of tested PD1-IL7 fusion proteins

| Description | IL7 variant | ID | SEQ ID NOs |
|---|---|---|---|
| Reference molecule 5 | D74E | P1AF9647-027 | 18, 20, 26 |
| Reference molecule 7 | SS3 | P1AF9649-012 | 18, 20, 28 |
| | (C47S/C92S, | | |
| | C34S/C129S) | | |
| Reference molecule 8 | W124H | P1AF9650-004 | 18, 20, 29 |

Cloning. The corresponding cDNAs were cloned into evitria's vector system using conventional (non-PCR based) cloning techniques. The evitria vector plasmids were gene synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with two sequencing reactions per plasmid.)

Production of IgG-like proteins in CHO cells. The antibody IL7 fusion constructs described herein were produced by Evitria using their proprietary vector system with conventional (non-PCR based) cloning techniques and using suspension-adapted CHO KI cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatants were harvested by centrifugation and subsequent filtration (0.2 μm filter).

Purification of IgG-like proteins. Proteins were purified from filtered cell culture supernatants referring to standard protocols. In brief, Fc containing proteins were purified from cell culture supernatants by Protein A-affinity chromatography (equilibration buffer: 20 mM sodium citrate, mM sodium phosphate, pH 7.5: elution buffer: 20 mM sodium citrate, pH 3.0). Elution was achieved at pH 3.0 followed by immediate pH neutralization of the sample. The protein was concentrated by centrifugation (Millipore Amicon® ULTRA-15: Art. Nr .: UFC903096), and aggregated protein was separated from monomeric protein by size exclusion chromatography in 20 mM histidine, 140 mM sodium chloride, pH 6.0.

Analytics of IgG-like proteins. The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCI pH 6.2, 0.02% NaN₃).

TABLE 5
Monomer product peak, high molecular weight (HMW) and
low molecular weight (LMW) side products determined
by analytical size exclusion chromatography (SEC).

| | | Monomer | HMW | LMW |
|---|---|---|---|---|
| | | peak | peak | peak |
| PD1-IL7 variant | ID | (%) | (%) | (%) |
| Reference molecule 5 | P1AF9647-027 | 97 | 1.9 | 1 |
| Reference molecule 7 | P1AF9649-012 | 94.1 | 1.9 | 4 |
| Reference molecule 8 | P1AF9650-004 | 99.1 | 0.9 | 0 |

TABLE 6
Main product peak determined by non-reduced CE-SDS.

| PD1-IL7 variant | ID | Main peak (%) |
|---|---|---|
| Reference molecule 5 | P1AF9647-027 | 99.11 |
| Reference molecule 7 | P1AF9649-012 | 91.7 |
| Reference molecule 8 | P1AF9650-004 | 94.54 |

Results. The purified PD1-IL7 variants constructs were purified by ProteinA and size exclusion chromatography. Reference molecule 7 was deglycosylated with PNGaseF prior to CE-SDS analysis to get a homogeneous peak. The quality analysis of the purified material revealed that the monomer content was above 94% by analytical size exclusion chromatography analysis (Table 5) and that the main product peak was between 91% and 99% by non-reduced capillary electrophoresis (Table 6). In conclusion, all PD1-IL7 variants were produced in good quality.

### Example 2

### Example 2. Affinity Determination of PD1-IL7 Variants to Human IL7 Receptor

TABLE 7
SPR running parameters

| | |
|---|---|
| Instrumentation | Biacore 8K (Cytiva) |
| Chip | C1 (# 903) |
| Fc1 to 8 | anti-P329G Fc specific IgG (Roche internal) |
| Capture | 5 nM PD1-IL7 variants for 140 s, 10 μl/min |
| Analyte | Two-fold serial dilution from 2.34 to 300 nM of hu |
| | IL7Rα- IL2Rγ-Fc avi biotin (heterodimer of the ECD of |
| | IL7Rα and IL2Rγ chains fused to an Fc) |
| Running buffer | HBS-EP (0.01M HEPES pH 7.4, 0.15M NaCl, 0.005% |
| | Surfactant P20 (BR-1006-69, Cytiva) + 1 mg/ml BSA |
| T° | 25° C. |
| Flow | 30 μl/min |
| Association | 240 sec |
| Dissociation | 800 sec |
| Regeneration | 10 mM glycine pH 2 for 2 x 60 sec |

SPR experiments were performed on a Biacore 8K with HBS-EP+1 mg/ml BSA as running buffer. Anti-P329G Fc specific antibody (Roche internal) was directly immobilized by amine coupling on a C1 chip (Cytiva). The PD1-IL7 constructs were captured for 140 s at 5 nM. Triplicates of a 2-fold serial dilution series from 2.34 to 300 nM human IL7Ra-IL2Rg-Fc heterodimer was passed over the ligand at 30 μl/min for 240 sec to record the association phase. The dissociation phase was monitored for 800 s and triggered by switching from the sample solution to running buffer. The chip surface was regenerated after every cycle using two injections of 10 mM glycine pH 2 for 60 sec. Bulk refractive index differences were corrected for by subtracting the response obtained on the reference flow cell (containing immobilized anti P329G Fc specific IgG only). The affinity constants were derived from the kinetic rate constants by fitting to a 1:1 Langmuir binding using the Biacore evaluation software (Cytiva).

Following PD1-IL7 variants were analyzed for binding to IL7 receptor (Table 8).

TABLE 8
Description of the samples analyzed for binding to IL7 receptor.

| PD1-IL7 variants | ID | Conc [g/l] |
|---|---|---|
| PD1-IL7wt | P1AF5572-018 | 4.4 |
| PD1(alt)-IL7-VAR21 | P1AG3439-001 | 1.35 |
| PD1(alt)-IL7-VAR18/VAR21 | P1AG3441-001 | 3.8 |
| Reference molecule 5 | P1AF9647-027 | 0.76 |
| Reference molecule 6 | P1AF9648-033 | 2.5 |
| Reference molecule 7 | P1AF9649-012 | 1.35 |
| Reference molecule 8 | P1AF9650-004 | 3.81 |
| Reference molecule 9 | P1AG8273-001 | 2.5 |
| Reference molecule 10 | P1AG8275-001 | 2.3 |
| Sample name analytes | ID | Conc [g/l] |
| human IL7Rα-IL2Rγ-Fc biotin | P1AF4984-007 | 1.43 |

Results. PD1(alt)-IL7 variants and reference molecules were compared for binding to human IL7 receptor (Table 9). The affinity of the IL7 variants to the IL7 receptor was determined using the recombinant heterodimer of the extracellular domains of the IL7 receptor alpha chain and the common IL2 receptor gamma chain fused to a human Fc.

TABLE 9
Binding of PD1-IL7 variants to human IL7 receptor: affinity
constants determined by surface plasmon resonance at 25°
C. Average of triplicates, standard deviation in parenthesis

| Sample name captured | | | | |
|---|---|---|---|---|
| molecules | ID | ka [1/Ms] | kd [1/s] | KD [M] |
| PD1-IL7 wt | P1AF5572-018 | 7.27E+05 | 2.47E−04 | 3.4E−10 |
| | | (2.21E+04) | (1.03E−05) | (1.4E−11) |
| PD1(alt)-IL7-VAR21 | P1AG3439-001 | 3.59E+05 | 2.13E−03 | 5.94E−09 |
| | | (1.91E+04) | (1.85E−04) | (6.13E−10) |
| PD1(alt)-IL7-VAR18/ | P1AG3441-001 | 8.05E+04 | 1.03E−02 | 1.29E−07 |
| VAR21 | | (7.84E+03) | (1.88E−03) | (2.93E−08) |
| Reference molecule 5 | P1AF9647-027 | 3.52E+05 | 2.37E−04 | 6.71E−10 |
| | | (6.51E+03) | (1.98E−05) | (4.38E−11) |
| Reference molecule 6 | P1AF9648-033 | 1.09E+05 | 1.08E−03 | 9.94E−09 |
| | | (4.04E+03) | | (9.24E−11) |
| Reference molecule 7 | P1AF9649-012 | very weak binding | | |
| Reference molecule 8 | P1AF9650-004 | 3.86E+05 | 3.27E−04 | 8.47E−10 |
| | | (2.51E+04) | (3.30E−05) | (6.66E−11) |
| Reference molecule 9 | P1AG8273-001 | 4.56E+05 | 3.16E−04 | 6.96E−10 |
| | | (1.96E+04) | (3.08E−05) | (9.33E−11) |
| Reference molecule 10 | P1AG8275-001 | 2.26E+05 | 1.10E−03 | 4.94E−09 |
| | | (1.59E+04) | (2.25E−04) | (1.29E−09) |

Both IL7 variants VAR21 (G85E) and VAR18/VAR21 (K81E, G85E) showed reduced affinity for the human IL7 receptor compared to wild-type IL7. The IL7 single mutant PD1(alt)-IL7 VAR21 binds to the human IL7 receptor with an affinity around 6 nM and the double mutant PD1(alt)-IL7 VAR18/VAR21 with an affinity 20-fold lower around 130 nM.

Reference molecules 5, 8 and 9 have a higher affinity to the human IL7 receptor (around 0.6 to 0.9 nM) and reference molecules 6 and 10 are close to PD1(alt)-IL7-VAR21 (G85E) with affinities of 10 and 5 nM respectively. Reference molecule 7 is hardly binding under these conditions and is considered inactive.

Conclusion. The mutations introduced in IL7 in PD1(alt)-IL7 VAR21 (G85E) and in PD1(alt)-IL7 VAR18/VAR21 (K81E, G85E) result in a reduced affinity to the human IL7 receptor compared to wild type IL7, with the double-mutant having an affinity 20-fold lower than the single mutant.

### Example 3.1 IL-7R Signaling (STAT5-P) on Activated PD-1⁺ and PD-1⁻ CD4 T Cells Upon Treatment With Increasing Doses of PD1(alt)-IL7 Variants

In this experiment, the cis-targeting and potency in STAT-5P signaling of the molecules PD1(alt)-IL7VAR21 and PD1(alt)-IL7VAR18/VAR21 were compared to PD1(alt)-IL7wt.

For this purpose the IL7R signaling was measured on PD1⁺ and PD1⁻ (anti-PD1 pre-treated) CD4 T cells, isolated, activated and co-cultured as previously described, after exposing the cells to increasing concentrations of immune-targeted cytokines.

The data in the FIG. 2 show the potency difference of PD1(alt)-IL7 VAR21, PD1(alt)-IL7 VAR18/VAR21 and PD1(alt)-IL7wt on PD-1⁺ and PD-1 pre-blocked CD4 T cells.

The potency measured on PD1⁺ CD4 T cells reflects the combination of PD1-dependent and independent delivery of IL-7. In contrast, the potency measurement on PD1 pre-blocked CD4 T cells represents the PD1-independent delivery of IL-7, as all the PD1 binding sites are occupied to prevent PD-1 binding.

The cis activity, the relation between PD1-dependent and independent delivery of IL-7, of each PD1(alt)-IL7v molecule was calculated in Table 10 by dividing the EC50 of the PD-1 pre-blocked cells by the EC50 of PD1³⁰ T cells. This provides a measurement of the strength of the PD1-dependent delivery of IL-7 for each of PD1(alt)-IL7 constructs, when the cells express the same level of IL-7Ra/common gamma chain.

TABLE 10
EC50, cis-activity, and fold reduction in potency of the dose-
response STAT-5 phosphorylation for the selected mutants on
PD-1+ and PD-1 pre-blocked CD4 T cells from healthy donors.

| | | | | Fold reduction in |
|---|---|---|---|---|
| | | | cis-activity as | potency as |
| | | | EC50 [PD1 | EC50 [PD1⁺] / |
| | | EC50 PD1 | pre-blocked] / | EC50 [PD1⁺ |
| PD1(alt)-IL7 variant | EC50 PD1⁺ | pre-blocked | EC50 [PD1⁺] | PD1(alt)-IL7wt] |
| PD1(alt)-IL7wt | 8.147 | 71.29 | 8.7 | 1.00 |
| PD1(alt)-IL7 VAR21 | 60.68 | 4876 | 80.35 | 7.5 |
| PD1(alt)-IL7 VAR18/VAR21 | 662 | nd | nd | 81.2 |

Although PD1(alt)-IL7 variants show a potency reduction on PD-1⁺ T cells when compared to PD1(alt)-IL7wt, they also display an even more pronounced reduced activity on PD-1⁻ T cells, with PD1(alt)-VAR18/VAR21 being virtually inactive.

### Example 3.2 IL-7R Signaling (STAT5-P) on Activated PD-1⁺ and PD-1⁻ CD4 T Cells Upon Treatment With Increasing Doses of PD1-IL7 Variants

In this experiment, the cis-targeting and potency in STAT-5P signaling of Reference molecules 5-10, generated by fusing mutated IL-7 to the blocking PD1 binder with the SEQ ID Nos: 3 and 4 were compared to the molecules PD1(alt)-IL7 VAR21 and PD1(alt)-IL7 VAR18/VAR21.

For this purpose the IL7R signaling was measured on PD1⁺ and PD1⁻ (anti-PD1 pre-treated) CD4 T cells, isolated, activated and co-cultured as previously described, after exposing the cells to increasing concentrations of immune-targeted cytokines.

CD4 T cells were sorted from healthy donor PBMCs with CD4 beads (130-045-101, Miltenyi) and activated for 3 days in presence of 1 μg/ml plate bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells were harvested and washed several times to remove endogenous IL-2. Then, the cells were divided in two groups, one of which was incubated with saturating concentration of anti-PD1 antibody (in-house molecule, 10 μg/ml) for 30 min at RT.

Following several washing steps to remove the excess unbound anti-PD-1 antibody, the anti-PD1 pre-treated and untreated cells (50 μl, 4*10⁶ cells/ml) were seeded into a V-bottom plate before being treated for 12min at 37° C. with increasing concentrations of treatment antibodies (50 μl, 1:10 dilution steps with the top concentration of 66 nM). To preserve the phosphorylation state, an equal amount of Phosphoflow Fix Buffer I (100 μl, 557870, BD) was added right after 12 minutes incubation with the various constructs. The cells were then incubated for additional 30 min at 37° C. before being permeabilized overnight at 80° C. with Phosphoflow PermBuffer III (558050, BD). On the next day STAT-5 in its phosphorylated form was stained for 30 min at 4° C. by using an anti-STAT-SP antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism.

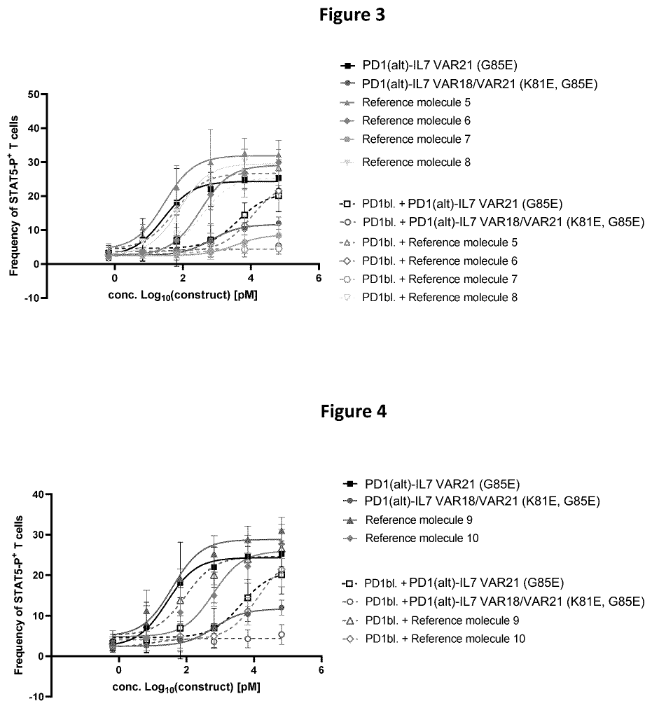

The data in the FIGS. 3 and 4 show the potency difference of PD1(alt)-IL7 VAR21, PD1(alt)-IL7 VAR18/VAR21 and reference molecules on PD-1⁺ and PD-1 pre-blocked CD4 T cells.

The potency measured on PD1⁺ CD4 T cells reflects the combination of PD1-dependent and independent delivery of IL-7. In contrast, the potency measurement on PD1 pre-blocked CD4 T cells represents the PD1-independent delivery of IL-7, as all the PD1 binding sites are occupied to prevent PD-1 binding.

The cis activity, the relation between PD1-dependent and independent delivery of IL-7, of each PD1-IL7y molecule was calculated in Table 11 by dividing the EC50 of the PD-1 pre-blocked cells by the EC50 of PD1⁺ T cells. This provides a measurement of the strength of the PD1-dependent delivery of IL-7 for each of PD1-IL7 constructs, when the cells express the same level of IL-7Ra/common gamma chain.

TABLE 11
EC50, cis-activity, and fold reduction in potency of the dose-
response STAT-5 phosphorylation for the selected mutants on
PD-1⁺ and PD-1 pre-blocked CD4 T cells from healthy donors.

| | | | | Fold reduction in |
|---|---|---|---|---|
| | | | cis-activity as | potency as EC50 |
| | | | EC50 [PD1 | [PD1⁺] / EC50 |
| | EC50 | EC50 PD1 | pre-blocked] / | [PD1⁺ PD1(alt)-IL7 |
| PD1-IL7 variant | PD1⁺ | pre-blocked | EC50 [PD1⁺] | VAR21] |
| PD1(alt)-IL7-VAR21 | 26.47 | 4177 | 157.8 | 1 |
| PD1(alt)-IL7-VAR/VAR21 | 676.2 | n.d. | n.d. | 25.5 |
| Reference molecule 5 | 29.04 | 58.74 | 2.0 | 1.1 |
| Reference molecule 6 | 329.3 | 10570 | 32.1 | 12.4 |
| Reference molecule 7 | 3518 | 461.1 | 0.1 | 133 |
| Reference molecule 8 | 79.32 | 355 | 4.5 | 3 |
| Reference molecule 9 | 37.64 | 94.45 | 2.5 | 1.42 |
| Reference molecule 10 | 621 | 12845 | 20.7 | 23.46 |

Although Reference molecule 5 and Reference molecule 9 are as potent as PD1(alt)-IL7 VAR21, both reference molecules show activity also on PD-1⁻ T cells, which is only 2 and 2.5 fold lower than on PD-1⁺ T cells, indicating a PD-1 independent delivery of the IL-7 variants, while PD1(alt)-IL7 VAR21 has roughly 160 fold lower activity on PD⁻¹ T cells.

Reference molecule 6 and Reference molecule 10 showed a 32-fold and 20-fold reduced activity, respectively, on PD-1⁻ T cells, when compared to PD-1⁺ T cells, supporting a PD-1 mediated cis delivery of IL-7R agonism, however both molecules showed a 12 and 23 fold reduced potency than PD1(alt)-IL7 VAR21 (Table 11, FIGS. 3 and 4).

PD1(alt)-IL7 VAR18/VAR21, which, although less potent and with a reduced maximal activity than PD1(alt)-IL7 VAR21, is virtually inactive on PD-1⁻ T cells demonstrating a strong cis-mediated delivery by PD-1 (FIGS. 3 and 4 and Table 11).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An immunoconjugate for use in the treatment of cancer, wherein the immunoconjugate comprises (i) a mutant IL-7 polypeptide and (ii) and antibody, wherein the antibody binds to PD1 and comprises the VH domain according to SEQ ID NO: 1 and the VL domain according to SEQ ID NO: 2; and wherein the mutant IL-7 polypeptide comprises an amino acid substitution at the position of G85 of human IL-7 according to SEQ ID NO: 14, wherein the amino acid substitution reduces the binding affinity of the mutant interleukin-7 polypeptide to IL-7Rα compared to an interleukin-7 polypeptide comprising SEQ ID NO: 14.

2. The immunoconjugate for use according to claim 1, wherein the amino acid substitution at the position of G85 of human IL-7 according to SEQ ID NO: 14 is G85E.

3. The immunoconjugate for use according to claim 1, wherein the mutant interleukin-7 polypeptide further comprises an amino acid substitution at position K81.

4. The immunoconjugate for use according to claim 1, wherein the mutant interleukin-7 polypeptide comprises the amino acid substitution K81E.

5. The immunoconjugate for use according to claim 1, wherein the immunoconjugate comprises not more than one mutant IL-7 polypeptide.

6. The immunoconjugate for use according to claim 1, wherein the antibody comprises an Fc domain composed of a first and a second subunit.

7. The immunoconjugate for use according to claim 6, wherein the Fc domain is an IgG class Fc domain.

8. The immunoconjugate for use according to claim 6, wherein the Fc domain is a human Fc domain.

9. The immunoconjugate for use according to claim 6, wherein the antibody is an IgG class immunoglobulin.

10. The immunoconjugate for use according to claim 6, wherein the Fc domain comprises a modification promoting the association of the first and the second subunit of the Fc domain.

11. The immunoconjugate for use according to claim 6, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

12. The immunoconjugate for use according to claim 6, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index).

13. The immunoconjugate for use according to claim 6, wherein in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

14. The immunoconjugate for use according to claim 6, wherein the mutant IL-7 polypeptide is fused at its amino-terminal amino acid to the carboxy-terminal amino acid of one of the subunits of the Fc domain, particularly the first subunit of the Fc domain, optionally through a linker peptide.

15. The immunoconjugate for use according to claim 14, wherein the linker peptide has the amino acid sequence of SEQ ID NO: 5.

16. The immunoconjugate for use according to claim 6, wherein the Fc domain comprises one or more amino acid substitution(s) that reduces binding to an Fc receptor and/or effector function.

17. The immunoconjugate for use according to claim 16, wherein said one or more amino acid substitution(s) is at one or more position(s) selected from the group consisting of L234, L235, and P329 (Kabat EU index numbering).

18. The immunoconjugate for use according to claim 6, wherein each subunit of the Fc domain comprises the amino acid substitutions L234A, L235A and P329G (Kabat EU index numbering).

19. The immunoconjugate for use according to claim 1, comprising a polypeptide comprising an amino acid sequence that is at least about 80%, 85%, 90%, 95%, 96%, 97%, 98%, 99% or 100% identical to the sequence of SEQ ID NO: 17, a polypeptide comprising an amino acid sequence that is at least about 80%, 85%, 90%, 95%, 96%, 97%, 98%, 99% or 100% identical to the sequence of SEQ ID NO: 19, and a polypeptide comprising an amino acid sequence that is at least about 80%, 85%, 90%, 95%, 96%, 97%, 98%, 99% or 100% identical to a sequence selected from the group consisting of SEQ ID NO: 24 and SEQ ID NO: 25.

20. A method of treating cancer in an individual, comprising administering to said individual a therapeutically effective amount of an immunoconjugate, wherein the immunoconjugate comprises (i) a mutant IL-7 polypeptide and (ii) and antibody, wherein the antibody binds to PD1 and comprises the VH domain according to SEQ ID NO: 1 and the VL domain according to SEQ ID NO: 2; and wherein the mutant IL-7 polypeptide comprises an amino acid substitution at the position of G85 of human IL-7 according to SEQ ID NO: 14, wherein the amino acid substitution reduces the binding affinity of the mutant interleukin-7 polypeptide to IL-7Rα compared to an interleukin-7 polypeptide comprising SEQ ID NO: 14.

21. The method according to claim 20, wherein the amino acid substitution at the position of G85 of human IL-7 according to SEQ ID NO: 14 is G85E.
